(19) United States
(12) Patent Application Publication
(10) Pub. No.: US 2026/0209378 A1
(43) Pub. Date: Jul. 23, 2026

(54) ANTIBODIES TO CUB DOMAIN-CONTAINING PROTEIN 1 (CDCP1) AND USES THEREOF

(71) Applicant: Pheon Therapeutics Ltd, Hertfordshire (GB)

(72) Inventors: Leigh Zawel, Hertfordshire (GB); Chi-Ting Huang, Hertfordshire (GB)

(21) Appl. No.: 19/138,599

(22) PCT Filed: Dec. 15, 2023
(86) PCT No.: PCT/IB2023/062818

§ 371 Date: Jun. 12, 2025

Related U.S. Application Data
(60) Provisional application No. 63/387,819, filed on Dec. 16, 2022.

(51) Int. Cl. C07K 16/28

(52) U.S. Cl. CPC C07K 16/2896; C07K 2317/33; C07K 2317/34; C07K 2317/565; C07K 2317/92

(57) ABSTRACT

The present disclosure provides antibodies and antigen binding fragments thereof that bind to human CDCP1. The disclosed antibodies are therefore useful for the treatment of cancer, either alone or in combination with other agents.

### CROSS-REFERENCE TO RELATED APPLICATIONS

This application claims priority to U.S. Provisional Application No. 63/387,819, filed Dec. 16, 2022, which is incorporated by reference herein in its entirety.

### SEQUENCE LISTING

The instant application contains a Sequence Listing that has been submitted electronically in XML file format and is hereby incorporated by reference in its entirety. The Sequence Listing for this application is labeled “133186-5016-SequenceListing.xml”, which was created on Nov. 27, 2023, and is 34,000 bytes in size.

### FIELD

The present disclosure relates to antibodies and antigen binding fragments thereof which bind to transmembrane glycoprotein, CUB domain-containing protein 1 (CDCP1). The disclosure further relates to the use of anti-CDCP1 antibodies and antigen binding fragments thereof for the treatment and detection of cancer.

### BACKGROUND

Human CDCP1 (also known as CUB domain containing protein 1, B345, CD318, SIMA135, TRASK) is a transmembrane protein containing three extracellular CUB domains and is widely expressed in human epithelial tissues. This protein is often phosphorylated and overexpressed in various cancers such as colon and lung cancers. Its expression level is correlated with the metastatic ability of carcinoma cells. Thus, there is a need to develop anti-CDCP1 antibodies that alone, or in combination with other for therapeutic agents can be used to target CDCP1 expressing cancer cells.

### SUMMARY

The present disclosure addresses the above need by providing anti-CDCP1 antibodies and antigen binding fragments thereof. These antibodies and antigen binding fragments thereof are characterized by a unique set of CDR sequences. Such antibodies and antigen binding fragments thereof may bind to human CDCP1 expressing cancer cells.

In one aspect, an anti-CDCP1 antibody or binding fragment thereof is provided that comprises:
(a) a heavy chain variable region comprising CDR1: SEQ ID NO: 3, CDR2: SEQ ID NO: 4, and CDR3: SEQ ID NO: 13; and/or a light chain variable region comprising CDR1: SEQ ID NO: 8, CDR2: SEQ ID NO: 9, and CDR3: SEQ ID NO: 10;
(b) a heavy chain variable region comprising CDR1: SEQ ID NO: 3, CDR2: SEQ ID NO: 4, and CDR3: SEQ ID NO: 16; and/or a light chain variable region comprising CDR1: SEQ ID NO: 8, CDR2: SEQ ID NO: 9, and CDR3: SEQ ID NO: 10;
(c) a heavy chain variable region comprising CDR1: SEQ ID NO: 3, CDR2: SEQ ID NO: 4, and CDR3: SEQ ID NO: 19; and/or a light chain variable region comprising CDR1: SEQ ID NO: 8, CDR2: SEQ ID NO: 9, and CDR3: SEQ ID NO: 10;
(d) a heavy chain variable region comprising CDR1: SEQ ID NO: 3, CDR2: SEQ ID NO: 4, and CDR3: SEQ ID NO: 22; and/or a light chain variable region comprising CDR1: SEQ ID NO: 8, CDR2: SEQ ID NO: 9, and CDR3: SEQ ID NO: 10;
(e) a heavy chain variable region comprising CDR1: SEQ ID NO: 3, CDR2: SEQ ID NO: 4, and CDR3: SEQ ID NO: 25; and/or a light chain variable region comprising CDR1: SEQ ID NO: 8, CDR2: SEQ ID NO: 9, and CDR3: SEQ ID NO: 10;
(f) a heavy chain variable region comprising CDR1: SEQ ID NO: 3, CDR2: SEQ ID NO: 4, and CDR3: SEQ ID NO: 28; and/or a light chain variable region comprising CDR1: SEQ ID NO: 8, CDR2: SEQ ID NO: 9, and CDR3: SEQ ID NO: 10; or
(g) a heavy chain variable region comprising CDR1: SEQ ID NO: 3, CDR2: SEQ ID NO: 4, and CDR3: SEQ ID NO: 31; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising one or more of the following amino acids mutations: (a) M103F; (b) M108L; (c) M103K; (d) M103L; (e) M103F and M108L; (f) M103K and M108L; or (g) M103L and M108L.

In some embodiments, the anti-CDCP1 antibody or binding fragment thereof comprises:
(a) a heavy chain variable (VH) region having an amino acid sequence as set forth in SEQ ID NO: 12 and a light chain variable (VL) region having an amino acid sequence as set forth in SEQ ID NO: 7;
(b) a heavy chain variable region having an amino acid sequence as set forth in SEQ ID NO: 15 and a light chain variable region having an amino acid sequence as set forth in SEQ ID NO: 7;
(c) a heavy chain variable region having an amino acid sequence as set forth in SEQ ID NO: 18 and a light chain variable region having an amino acid sequence as set forth in SEQ ID NO: 7;
(d) a heavy chain variable region having an amino acid sequence as set forth in SEQ ID NO: 21 and a light chain variable region having an amino acid sequence as set forth in SEQ ID NO: 7;
(e) a heavy chain variable region having an amino acid sequence as set forth in SEQ ID NO: 24 and a light chain variable region having an amino acid sequence as set forth in SEQ ID NO: 7;
(f) a heavy chain variable region having an amino acid sequence as set forth in SEQ ID NO: 27 and a light chain variable region having an amino acid sequence as set forth in SEQ ID NO: 7; or
(g) a heavy chain variable region having an amino acid sequence as set forth in SEQ ID NO: 30 and a light chain variable region having an amino acid sequence as set forth in SEQ ID NO: 7.

In some embodiments, the anti-CDCP1 antibody or binding fragment thereof comprises a heavy chain constant region comprising the amino acid sequence set forth in SEQ ID NO: 32. In some embodiments, the anti-CDCP1 antibody or binding fragment thereof comprise a light chain constant region comprising the amino acid sequence set forth in SEQ ID NO: 33.

In some embodiments, the anti-CDCP1 antibody or binding fragment thereof comprises a heavy chain variable region comprising CDR1: SEQ ID NO: 3, CDR2: SEQ ID NO: 4 and CDR3: SEQ ID NO: 13; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising CDR1: SEQ ID NO: 3, CDR2: SEQ ID NO: 4, and CDR3: SEQ ID NO: 16; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising CDR1: SEQ ID NO: 3, CDR2: SEQ ID NO: 4, and CDR3: SEQ ID NO: 19; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising CDR1: SEQ ID NO: 3, CDR2: SEQ ID NO: 4, and CDR3: SEQ ID NO: 22; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising CDR1: SEQ ID NO: 3, CDR2: SEQ ID NO: 4, and CDR3: SEQ ID NO: 25; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising CDR1: SEQ ID NO: 3, CDR2: SEQ ID NO: 4, and CDR3: SEQ ID NO: 28; and/or a light chain variable region comprising CDR1: SEQ ID NO: 8, CDR2: SEQ ID NO: 9, and CDR3: SEQ ID NO: 10.

In some embodiments, the anti-CDCP1 antibody or binding fragment thereof comprises a heavy chain variable region comprising CDR1: SEQ ID NO: 3, CDR2: SEQ ID NO: 4, and CDR3: SEQ ID NO: 31; and/or a light chain variable region comprising CDR1: SEQ ID NO: 8, CDR2: SEQ ID NO: 9, and CDR3: SEQ ID NO: 10.

According to some embodiments, the anti-CDCP1 antibody or binding fragment thereof comprises a set of six complementarity determining region (CDR) sequences selected from the group consisting of three CDRs of a heavy chain (HC) variable region selected from SEQ ID NOs: 12, 15, 18, 21, 24, 27 and 30, and/or three light CDRs of a light chain (LC) variable region of SEQ ID NO: 7. In an embodiment, the anti-CDCP1 antibody or binding fragment thereof comprises a heavy chain and/or light chain having at least 90%, 95%, or 99% sequence identity with the heavy chain and/or light chain of any of the anti-CDCP1 antibodies or binding fragments disclosed herein.

In some embodiments, the anti-CDCP1 antibody or binding fragment thereof comprises a heavy chain variable region sequence selected from the group consisting of SEQ ID NOs: 12, 15, 18, 21, 24, 27 and 30.

In other embodiments, the anti-CDCP1 antibody or binding fragment thereof comprises a light chain variable region sequence of SEQ ID NO: 7.

In other embodiments, the anti-CDCP1 antibody or binding fragment thereof comprises a heavy chain variable region sequence selected from the group consisting of SEQ ID NOs: 12, 15, 18, 21, 24, 27 and 30 and a light chain variable region sequence of SEQ ID NO: 7. In some embodiments, the anti-CDCP1 antibody or binding fragment thereof comprises a heavy chain sequence selected from the group consisting of SEQ ID NOs: 11, 14, 17, 20, 23, 26 and 29.

In other embodiments, the anti-CDCP1 antibody or binding fragment thereof comprises a light chain sequence of SEQ ID NO: 6.

In other embodiments, the anti-CDCP1 antibody or binding fragment thereof comprises a heavy chain sequence selected from the group consisting of SEQ ID NOs: 11, 14, 17, 20, 23, 26 and 29 and a light chain sequence of SEQ ID NO: 6.

In some embodiments, the anti-CDCP1 antibody or binding fragment thereof comprises a heavy chain variable region sequence and a light chain variable region sequence, selected from the following:
(a) a heavy chain variable region sequence comprising SEQ ID NO: 11 and a light chain variable region sequence comprising SEQ ID NO: 6;
(b) a heavy chain variable region sequence comprising SEQ ID NO: 14 and a light chain variable region sequence comprising SEQ ID NO: 6;
(c) a heavy chain variable region sequence comprising SEQ ID NO: 17 and a light chain variable region sequence comprising SEQ ID NO: 6;
(d) a heavy chain variable region sequence comprising SEQ ID NO: 20 and a light chain variable region sequence comprising SEQ ID NO: 6;
(e) a heavy chain variable region sequence comprising SEQ ID NO: 23 and a light chain variable region sequence comprising SEQ ID NO: 6;
(f) a heavy chain variable region sequence comprising SEQ ID NO: 26 and a light chain variable region sequence comprising SEQ ID NO: 6; and
(g) a heavy chain variable region sequence comprising SEQ ID NO: 29 and a light chain variable region sequence comprising SEQ ID NO: 6.

In some embodiments, the anti-CDCP1 antibody is a fully human antibody. In some embodiments, the anti-CDCP1 antibody is a chimeric antibody. In some embodiments, the anti-CDCP1 antibody is a bispecific or multispecific antibody. In some embodiments, the anti-CDCP1 antibody is a humanized antibody.

In some embodiments, the anti-CDCP1 antibody is an antibody fragment. In some embodiments, the antibody fragment is selected from the group consisting of: Fab, Fab, F(ab)2, Fd, Fv, scFv and scFv-Fc fragment, a single-chain antibody, a minibody, and a diabody.

In another aspect, an anti-CDCP1 antibody or binding fragment thereof is provided that comprises:
a1) a heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 13 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a2) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 16 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a3) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 19 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a4) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 22 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a5) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 25 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a6) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 28 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a7) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 31 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
or
b1) a heavy chain variable (VH) region comprising an amino acid sequence at least 90%, 95%, or 99% identical to SEQ ID NOs: 12, 15, 18, 21, 24, 27, or 30 and
b2) a light chain variable (VL) region comprising an amino acid sequence at least 90%, 95%, or 99% identical to SEQ ID NO: 7.

Also provided herein are antibody drug conjugates (ADCs) that comprise an anti-CDCP1 antibody or binding fragment thereof disclosed herein.

The present disclosure also provides pharmaceutical compositions comprising or consisting of at least one of the antibodies or antigen binding fragments thereof disclosed herein, and optionally a pharmaceutically acceptable diluent, carrier, vehicle and/or excipient. Such a pharmaceutical composition may be used for of the treatment of cancer. In some embodiments, the pharmaceutical composition is used in modulating the immune system by blocking or inhibiting CDCP1/EGFR and/or CDCP1/HER2 binding interactions.

The present disclosure also provides methods for the treatment of cancer in a patient comprising administering to the patient a therapeutically effective amount of an anti-CDCP1 antibody or binding fragment thereof as disclosed herein. Such antibodies or antigen binding fragments thereof may be administered alone or in combination with another therapeutic agent.

In one aspect, provided herein is a method of treating cancer comprising administering to a subject in need thereof, a pharmaceutical composition as disclosed herein.

In one aspect, a composition of isolated polynucleotides is provided. The composition of isolated polynucleotides may comprise: a first nucleic acid sequence encoding the heavy chain variable region of an anti-CDCP1 antibody as disclosed herein and a second nucleic acid sequence encoding the light chain variable region of an anti-CDCP1 antibody as disclosed herein. In some embodiments, the first nucleic acid sequence encodes an amino acid sequence as set forth in any one of SEQ ID NOS: 12, 15, 18, 21, 24, 27 and 30. In some embodiments, the second nucleic acid sequence encodes an amino acid sequence as set forth in SEQ ID NO: 7.

In some embodiments, the anti-CDCP1 antibody or binding fragment thereof comprises a heavy chain constant region comprising the amino acid sequence set forth in SEQ ID NO: 32.

In one aspect, a composition of isolated polynucleotides is provided. The composition of isolated polynucleotides may comprise: a first nucleic acid sequence encoding the heavy chain region of an anti-CDCP1 antibody as disclosed herein and a second nucleic acid sequence encoding the light chain of an anti-CDCP1 antibody as disclosed herein. In some embodiments, the first nucleic acid sequence encodes an amino acid sequence as set forth in any one of SEQ ID NOs: 11, 14, 17, 20, 23, 26 and 29. In some embodiments, the second nucleic acid sequence encodes an amino acid sequence as set forth in SEQ ID NO: 6.

In one aspect, provided herein is a vector comprising the disclosed polynucleotides. In some embodiments, the vector is an expression vector.

In another aspect, provided herein is a host cell or a vector comprising the disclosed nucleic acids. In some embodiments, at least two vectors are provided. In some embodiments, provided herein is a first vector comprising a first nucleic acid sequence encoding the heavy chain variable region of an anti-CDCP1 antibody as disclosed herein. In some embodiments, provided herein is a second vector comprising a second nucleic acid sequence encoding the light chain variable region of an anti-CDCP1 antibody as disclosed herein. In some embodiments, the first vector comprises a first nucleic acid sequence encoding an amino acid sequence as set forth in any one of SEQ ID NOS: 12, 15, 18, 21, 24, and 27. In some embodiments, the second vector comprises a second nucleic acid sequence encoding an amino acid sequence as set forth in SEQ ID NO: 7.

In another aspect, provided herein is a host cell or a vector comprising the disclosed nucleic acids. In some embodiments, at least two vectors are provided. In some embodiments, provided herein is a first vector comprising a first nucleic acid sequence encoding the heavy chain of an anti-CDCP1 antibody as disclosed herein. In some embodiments, provided herein is a second vector comprising a second nucleic acid sequence encoding the light chain of an anti-CDCP1 antibody as disclosed herein. In some embodiments, the first vector comprises a first nucleic acid sequence encoding an amino acid sequence as set forth in any one of SEQ ID NOs: 11, 14, 17, 20, 23, 26 and 29. In some embodiments, the second vector comprises a second nucleic acid sequence encoding an amino acid sequence as set forth in SEQ ID NO: 6.

In some embodiments, a host cell is provided that comprises one or more of the expression vectors provided herein.

In some embodiments, a host cell comprising the disclosed isolated polynucleotide is also provided.

In yet another aspect, provided herein is a method for the production of the disclosed anti-CDCP1 antibody or binding fragment thereof. In embodiments, the method comprises culturing the host cell disclosed herein in a medium and recovering the anti-CDCP1 antibody or binding fragment thereof from the medium.

### BRIEF DESCRIPTION OF THE OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A-1H are series of tables depicting the amino acid sequences of the heavy chain and light chain of a reference anti-CDCP1 antibody and its mutants (SEQ ID NOs: 1, 6, 11, 14, 17, 20, 23, 26 and 29). The amino acid sequences of the heavy and light constant and variable regions as well as the corresponding CDRs (CDR1, CDR2 and CDR3) are also provided (SEQ ID NOs: 2-5, 7-10, 12, 13, 15, 16, 18, 19, 21, 22, 24, 25, 27, 28, 30, 31 and 32). Reference methionine and mutants (F, L, and/or K) of VH CDR3 are highlighted in light grey. FIG. 1A: Reference anti-CDCP1; FIG. 1B: anti-CDCP1 mutant M103 FIG. 1C: anti-CDCP1 mutant M103F/M108L; FIG. 1D: anti-CDCP1 mutant M103K; FIG. 1E: anti-CDCP1 mutant M103K/M108L; FIG. 1F: anti-CDCP1 mutant M103L; FIG. 1G: anti-CDCP1 mutant M103L/M108L; and, FIG. 1H: anti-CDCP1 mutant M108L.

### DETAILED DESCRIPTION

The present disclosure relates to antibodies and antigen binding fragments thereof which bind to transmembrane glycoprotein, CUB domain-containing protein 1 (CDCP1). The disclosure further relates to several anti-CDCP1 antibodies variants and antigen binding fragments thereof. Advantageously, the disclosed anti-CDCP1 antibodies variants and antigen binding fragments thereof can be used for the treatment and detection of cancer.

So that the disclosure may be more readily understood, certain technical and scientific terms are specifically defined below. Unless specifically defined elsewhere in this document, all other technical and scientific terms used herein have the meaning commonly understood by one of ordinary skill in the art to which this disclosure belongs.

### Definitions

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

As used herein the term “antibody-drug conjugate” (ADC) refers to immunoconjugates consisting of recombinant monoclonal antibodies covalently linked to cytotoxic agents (known as payloads) via synthetic linkers. Immunoconjugates (antibody-drug conjugates, ADCs) are a class of highly potent antibody-based cancer therapeutics. ADCs consist of recombinant monoclonal antibodies covalently linked to cytotoxic agents (known as payloads) via synthetic linkers. ADCs combine the specificity of monoclonal antibodies and the potency of small-molecule chemotherapy drugs and facilitate the targeted delivery of highly cytotoxic small molecule drug moieties directly to tumor cells.

The term “affinity,” as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Throughout this disclosure the following abbreviations will be used:
mAb or Mab or MAb-Monoclonal antibody.
CDR—Complementarity determining region in the immunoglobulin variable regions.
VH—Immunoglobulin heavy chain variable region.
VL—Immunoglobulin light chain variable region.
FR—Antibody framework region, the immunoglobulin variable regions excluding the CDR regions.

The terms “CUB domain-containing protein 1 antibody” or “anti-CDCP1 antibody”, used interchangeably herein, refer to an antibody which binds to or binds specifically to CDCP1, e.g., human CDCP1 (e.g., the amino acid sequence provided in GenBank Accession No. NP 073753.3 and/or the amino acid sequence provided in NP_835488.1, the entire contents of each of which are incorporated herein by reference). An antibody “which binds” an antigen of interest, i.e., CDCP1, is one capable of binding that antigen with sufficient affinity such that the antibody is useful in targeting a cell expressing the antigen. In a preferred embodiment, the antibody specifically binds to human CDCP1 (hCDCP1). Examples of anti-CDCP1 antibodies are disclosed in the Examples, below. Unless otherwise indicated, the term “anti-CDCP1 antibody” is meant to refer to an antibody which binds to reference CDCP1, a variant, or an isoform of CDCP1.

Alternative splicing results in at least two transcript variants of hCDCP1. CDCP1 nucleotide and polypeptide sequences are reported as Accession Nos. NM 022842.4 (transcript variant 1 mRNA), NP_073753.3 (isoform 1 polypeptide) and NM_178181.2 (transcript variant 2 mRNA), NP_835488.1 (isoform 2 polypeptide). CDCP1 contains a CUB domain at positions 225-297 of NP_073753.3. The extracellular domain of the protein described in NP_073753.3 includes amino acid residues 30-667.

The terms “specific binding” or “specifically binding”, as used herein, in reference to the interaction of a CDCP1 antibody with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target.

As used herein the term “specifically binds CDCP1” refers to the ability of an antibody, or antigen-binding fragment to recognize and bind endogenous human CDCP1 as it occurs on the surface of normal or malignant cells and to recombinant cells engineered to overexpress human CDCP1 either stably or transiently. In one embodiment, the ability of an anti-CDCP1 antibody to interact with CDCP1 (human or cynomolgus monkey CDCP1) has a dissociation constant (Kn) of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less. In another embodiment, the phrase “specifically binds to hCDCP1” or “specific binding to hCDCP1”, as used herein, refers to the ability of an anti-CDCP1 antibody to interact with hCDCP1 with a dissociation constant (Kn) of between about 1 pM (0.001 nM) to 2,000 nM, between about 500 μM (0.5 nM) to 1,000 nM, between about 500 μM (0.5 nM) to 500 nM, between about 1 nM) to 200 nM, between about 1 nM to 100 nM, between about 1 nM to 50 nM, between about 1 nM to 20 nM, or between about 1 nM to 5 nM. In one embodiment, Kn is determined by surface plasmon resonance or Bio-Layer Interferometry, or by any other method known in the art. Bio-Layer Interferometry refers to an optical phenomenon that allows for the analysis of real-time biospecific interactions by measuring the interference patterns of reflected white light, for example using the Octet™ system (ForteBio, Pall Corp. Fremont, CA). For further description of the Octet™ system, see Li, B et al. (2011) J. Pharm. Biomed. Anal. 54 (2): 286-294 and Abdiche, Y. N., et al. (2009) Anal. Biochem. 386 (2): 172-180, the contents of which are incorporated herein by reference.

The term “antibody” broadly refers to an immunoglobulin (lg) molecule, generally comprised of four polypeptide chains, two heavy (H) chains and two light (L) chains, or any functional fragment, mutant, variant, or derivative thereof, that retains the essential target binding features of an lg molecule. Such mutant, variant, or derivative antibody formats are known in the art. Non-limiting embodiments of which are discussed below. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY) and class (e.g., IgG1, IgG2, IgG 3, IgG4, IgAQ1 and IgA2) or subclass. The term “antibody” herein is used in the broadest sense and encompasses various antibody structures, including but not limited to monoclonal antibodies, polyclonal antibodies, and multispecific antibodies (e.g., bispecific antibodies). The term “antigen binding portion” or “binding fragment thereof” of an antibody (or simply “antibody portion”), as used herein, refers to one or more fragments of an antibody that retain the ability to specifically bind to an antigen (e.g., hCDCP1). It has been shown that the antigen binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen binding portion” of an antibody include (i) a Fab fragment, a monovalent fragment consisting of the VL, VH, CL and CHI domains; (ii) a F(ab′h fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546, Winter et al., PCT publication WO 90/05144 Al herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding portion” of an antibody. In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more the binding fragments disclosed herein linked to a linker polypeptide or an immunoglobulin constant domain. Linker polypeptides comprise two or more amino acid residues joined by peptide bonds and are used to link one or more binding fragments. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Antibody portions, such as Fab and F(ab′h fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

As used herein, the term “portion” refers to the minimal number of amino acids or nucleic acids, as appropriate, to constitute an immunogenic epitope of the antigen of interest. An “isolated antibody”, as used herein, is intended to refer to an antibody that is substantially free of other antibodies having different antigenic specificities (e.g., an isolated antibody that specifically binds CDCP1 is substantially free of antibodies that specifically bind antigens other than CDCP1). An isolated antibody that specifically binds CDCP1 may, however, have cross-reactivity to other antigens, such as CDCP1 molecules from other species. Moreover, an isolated antibody may be substantially free of other cellular material and/or chemicals. The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87, 2007. In a preferred embodiment, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain.

The terms “monoclonal antibody” or “mAb” as used herein refers to an antibody obtained from a population of substantially homogeneous antibodies, e.g., the individual antibodies comprising the population are identical and/or bind the same epitope, except for possible variant antibodies, e.g., containing naturally occurring mutations or arising during production and/or storage of a monoclonal antibody preparation. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric antibody” refers to a recombinant antibody in which a portion of the heavy and/or light chain is identical with or homologous to corresponding sequences in antibodies derived from a particular species, or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. In addition, complementarity determining region (CDR) grafting may be performed to alter certain properties of the antibody molecule including affinity or specificity. Typically, the variable domains are obtained from an antibody from an experimental animal (the “parental antibody”), such as a rodent, and the constant domain sequences are obtained from human antibodies, so that the resulting chimeric antibody can direct effector functions in a human subject and will be less likely to elicit an adverse immune response than the parental (e.g., mouse) antibody from which it is derived.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. In particular, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. Humanized antibodies are typically less immunogenic to humans, relative to non-humanized antibodies, and thus offer therapeutic benefits in certain situations. Those skilled in the art will be aware of humanized antibodies and will also be aware of suitable techniques for their generation. See for example, Hwang, W. Y. K., et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and Selick et al., WO 90/07861, each of which is incorporated herein by reference in its entirety.

A “human antibody” is an antibody that possesses an amino-acid sequence corresponding to that of an antibody produced by a human and/or has been made using any of the techniques for making human antibodies known to one of skill in the art. This definition of a human antibody specifically excludes a humanized antibody comprising non-human antigen-binding residues. Human antibodies can be produced using various techniques known in the art, including methods described in Cole et al, Monoclonal Antibodies and Cancer Therapy, Alan R. Liss, p. 77 (1985); Boerner et al, J. Immunol, 147 (I): 86-95 (1991). See also van Dijk and van de Winkel, Curr. Opin. Pharmacol, 5:368-74 (2001). Human antibodies can be prepared by administering the target antigen to a transgenic animal that has been modified to produce such antibodies in response to antigenic challenge, but whose endogenous loci have been disabled, e.g., immunized HuMab mice (see, e.g., Nils Lonberg et al., 1994, Nature 368:856-859, WO 98/24884, WO 94/25585, WO 93/1227, WO 92/22645, WO 92/03918 and WO 01/09187 regarding HuMab mice), xenomice (see, e.g., U.S. Pat. Nos. 6,075,181 and 6,150,584 regarding XENOMOUSE™ technology) or Trianni mice (see, e.g., WO 2013/063391, WO 2017/035252 and WO 2017/136734).

The term “multispecific antibody” is used in the broadest sense and specifically covers an antibody comprising a heavy chain variable domain (VH) and a light chain variable domain (VL), where the VH-VL unit has polyepitopic specificity (e.g., is capable of binding to two different epitopes on one biological molecule or each epitope on a different biological molecule). Such multispecific antibodies include, but are not limited to, full-length antibodies, antibodies having two or more VL and VH domains, bispecific diabodies and triabodies. “Polyepitopic specificity” refers to the ability to specifically bind to two or more different epitopes on the same or different target(s).

“Dual specificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody in an IgG1 form binds to each epitope with an affinity of 5 μM to 0.001 μM, 3 μM to 0.001 μM, 1 μM to 0.001 μM, 0.5 μM to 0.001 μM or 0.1 μM to 0.001 μM. “Monospecific” refers to the ability to bind only one epitope. Multi-specific antibodies can have structures similar to full immunoglobulin molecules and include Fc regions, for example IgG Fc regions. Such structures can include, but are not limited to, IgG-Fv, IgG-(scFv) 2, DVD-Ig, (scFv) 2-(scFv) 2-Fc and (scFv) 2-Fc-(scFv) 2. In case of IgG-(scFv) 2, the scFv can be attached to either the N-terminal or the C-terminal end of either the heavy chain or the light chain.

As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fe), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain. The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well-known in the art.

The terms “Kabat numbering,” “Kabat definitions,” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NYAcad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “complementarity determining region” or “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework (FR)” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), Vols. 1-3. In one embodiment, for the VL, the subgroup is subgroup kappa I as in Kabat et al., supra. In one embodiment, for the VH, the subgroup is subgroup Ill as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The terms “Fc region” and “constant region” are used herein to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, the numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991). As used herein, the term “non-native constant region” refers to an antibody constant region that is derived from a source that is different from the antibody variable region or that is a human-generated synthetic polypeptide having an amino sequence that is different from the native antibody constant region sequence. For instance, an antibody containing a non-native constant region may have a variable region derived from a non-human source (e.g., a mouse, rat, or rabbit) and a constant region derived from a human source (e.g., a human antibody constant region), or a constant region derived from another primate, (e.g., pig, goat, rabbit, hamster, cat, dog, guinea pig, member of the bovidae family (such as cattle, bison, buffalo, elk, and yaks, among others), cow, sheep, horse, or bison, among others). The term “endogenous” describes a molecule (e.g., a polypeptide, nucleic acid or cofactor) that is found naturally in a particular organism (e.g., a human) or in a particular location within an organism (e.g., a tissue, organ, or a cell) such as CDCP1 transmembrane protein expressed by human cells.

The term “effector function(s),” deriving from the interaction of an antibody Fc region with certain Fc receptors, include but are not necessarily limited to Clq binding, complement dependent cytotoxicity (CDC), Fc receptor binding, FcyR-mediated effector functions such as ADCC and antibody dependent cell-mediated phagocytosis (ADCP), and down regulation of a cell surface receptor. Such effector functions generally require the Fc region to be combined with an antigen binding domain (e.g., an antibody variable domain). The term “Fc receptor” or “FcR” describes an antibody receptor that binds to the Fc region of an immunoglobulin, which is involved in antigen recognition located at the membrane of certain immune cells including B lymphocytes, natural killer cells, macrophages, neutrophils, and mast cells. Fc receptors recognizing the Fc portion of IgG are called Fc gamma receptors (FcγRs). The FcγR family includes allelic variants and alternatively spliced forms of these receptors. Based on the differences in structure, function, and affinity for IgG binding, FcγRs are classified into three major groups: FcγRI, FcγRII (FcγRIIa and FcγRIIb) and FcγRIII (FcγRIIIa and FcγRIIIb). Among them, FcγRI (CD64), FcγRIIa (CD32a), and FcγRIIIa (CD16a) are activating receptors containing the signal transduction motif, immunoreceptor tyrosine-based activation motif (ITAM), in the γ subunit of FcγRI and FcγRIIIa, or in the cytoplasmic tail of FcγRIIa. After binding of antigen-antibody complexes the activatory Fcγ receptors (human: FcγRI, FcγRIIA, FcγRIIC, FcγRIIIA, FcγRIIIB and murine: FcγRI, FcγRIII, FcγRIV) trigger immune effector functions. In contrast, FcγRIIb (CD32b) is an inhibitory receptor. Cross-linking of FcγRIIb leads to the phosphorylation of the immunoreceptor tyrosine-based inhibitory motif (ITIM) and inhibitory signaling transduction (Patel et al. Front Immunol. 2019; 10:223.).

As used herein, the terms “T regulatory cell,” or “Treg” refers to a cell of the immune system that have a regulatory role by suppressing/inhibiting the proliferation, activation and cytotoxic capacity of other immune cells such as CD8 positive (CD8+) effector T cells. Regulatory T cells (Tregs) are characterized by the expression of the master transcription factor forkhead box P3 (Foxp3). There are two major subsets of Treg cells, “natural” Treg (nTreg) cells that develop in the thymus, and “induced” Treg (iTreg) cells that arise in the periphery from CD4+ Foxp3− conventional T cells. Natural Tregs are characterized as expressing both the CD4 T cell co-receptor and CD25, which is a component of the IL-2 receptor. Treg are thus CD4+CD25+. Expression of the nuclear transcription factor Forkhead box P3 (FoxP3) is the defining property which determines natural Treg development and function. Treg cells exert their suppressive effects by numerous modes of action including suppression by: secretion of inhibitory cytokines (e.g., IL-10, TGFβ, IL-35), modulation of dendritic cell function/maturation, expression of immunoregulatory surface molecules (e.g., CTLA-4, LAG-3) or cytolysis (e.g., granzyme A- and or B-mediated).

As used herein, the term “proliferation” in the context of a population of cells, such as a population of CDCP1+ cells (e.g., T-regs, MDSCs, or CDCP1+ cancer cells) refers to mitotic and cytokinetic division of a cell so as to produce a plurality of cells. Cell proliferation may be evidenced, for example, by a finding that the quantity of cell (e.g., CDCP1+ cells) in a sample of cells has increased over a given time period, such as over the course of one or more days. In the present disclosure, cell proliferation is considered to be “inhibited” when the rate of a population of cells, such as a population of CDCP1+ cells contacted with an antagonistic anti-CDCP1 antibody described herein, is decreased relative to the proliferation of a population of control cells, such as a population of CDCP1 cells not contacted with the antagonistic anti-CDCP1 antibody.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more. The amino acid residues of an antibody that contact an antigen can be determined, for example, by determining the crystal structure of the antibody in complex with the antigen or by performing hydrogen/deuterium exchange. In some embodiments, residues of an antibody that are within 5 Å the antigen are considered to contact the antigen. In some embodiments, an antibody that binds to the same epitope as a reference antibody blocks binding of the reference antibody to its antigen in a competition assay by 50% or more, and conversely, the reference antibody blocks binding of the antibody to its antigen in a competition assay by 50% or more.

The term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab)₂; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CH1). Pepsin treatment of an antibody yields a single large F(ab)₂ fragment which roughly corresponds to two disulfide linked Fab fragments having divalent antigen-binding activity and is still capable of cross-linking antigen. Fab fragments differ from Fab′ fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, CL and CH domains; (ii) F (ab′) 2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., Nature 341:544-546, 1989), which consist of a VH domain; (vi) isolated complementarity determining regions (CDR), and (vii) combinations of two or more isolated CDRs which may optionally be joined by a synthetic linker.

As used herein, the term “diabodies” refers to bivalent antibodies comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. In one embodiment, the disclosure includes an amino acid sequence having at least 80%, at least 85%, at least 90%, at least 95%, at least 96%, at least 97%, at least 98%, or at least 99% identity to an amino acid sequence set forth in any one of SEQ ID NOs: 1 to 400 and 402-505.

The term “activity” includes activities such as the binding specificity/affinity of an antibody for an antigen, for example, an anti-hCDCP1 antibody that binds to a CDCP1 antigen. In one embodiment, an anti-CDCP1 antibody or anti-CDCP1 activity includes, but it not limited to, binding to CDCP1 in vitro; binding to CDCP1 on cells expressing CDCP1 in vivo; modulating (e.g., inhibiting) src and/or EGFR signaling; inducing cell death in cells expressing CDCP1, including breast cancer cells, e.g., triple negative breast cancer cells, colon cancer cells, lung cancer cells, e.g., non-small cell lung carcinoma (NSCLC) cells, liver cancer cells, pancreatic cancer cells, ovarian cancer cells, kidney cancer cells; inhibiting cancer cell invasion and metastasis; decreasing or inhibiting cancer, e.g., breast cancer, e.g., triple negative breast cancer, colon cancer, lung cancer, e.g., non-small cell lung carcinoma (NSCLC), liver cancer, pancreatic cancer, ovarian cancer, kidney cancer; and decreasing or inhibiting tumor cellular proliferation or tumor growth in vivo. In one embodiment, an anti-CDCP1 antibody is capable of being internalized into a cell expressing CDCP1 and/or inducing cytotoxicity.

The term “epitope” refers to a region of an antigen that is bound by an antibody, antibody fragment. As described by (Janeway, C, Jr., P. Travers, et al. (2001). Immunobiology: the immune system in health and disease. Part II, Section 3-8. New York, Garland Publishing, Inc.): “An antibody generally recognizes only a small region on the surface of a large molecule such as a protein . . . [Certain epitopes] are likely to be composed of amino acids from different parts of the [antigen] polypeptide chain that has been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al. (2001). Immunobiology: the immune system in health and disease. Part II, Section 3-8. New York, Garland Publishing, Inc.). In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

The term “Kd”, as used herein, refers to the equilibrium dissociation constant, which is obtained from the ratio of kd to ka (i.e., kd/ka) and is expressed as a molar concentration (M). Kd values for antibodies can be determined using methods well established in the art. Preferred methods for determining the Kd of an antibody include biolayer interferometry (BLI) analysis, preferably using a Fortebio Octet RED device, surface plasmon resonance, preferably using a biosensor system such as a BIACORE® surface plasmon resonance system, or flow cytometry and Scatchard analysis.

“EC₅₀” with respect to an agent and a particular activity (e.g. binding to a cell, inhibition of enzymatic activity, activation or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

The term “tumor microenvironment” refers to cancer cells that form a tumor and the population of non-cancer cells, molecules, and/or blood vessels within the tumor or that border or surround the cancer cells.

As used herein the terms “antibody-based immunotherapy” and “immunotherapies” are used to broadly refer to any form of therapy that relies on the targeting specificity of an anti-CDCP1 antibody, bispecific molecule, antigen-binding domain, or fusion protein comprising an anti-CDCP1 antibody or antibody fragments or CDRs thereof, to mediate a direct or indirect effect on a CDCP1 expressing cell. The terms are meant to encompass methods of treatment using naked antibodies, bispecific antibodies (including T cell engaging, NK cell engaging and other immune cell/effector cell engaging formats) antibody drug conjugates, cellular therapies using T cells (CAR-T) or NK cells (CAR-NK) engineered to comprise a CDCP1-specific chimeric antigen receptor and oncolytic viruses comprising a CDCP1 specific binding agent, and gene therapies by delivering the antigen binding sequences of the anti-CDCP1 antibodies and express the corresponding antibody fragments in vivo.

The term “competitive binding”, as used herein, refers to a situation in which a first antibody competes with a second antibody, for a binding site on a third molecule, e.g., an antigen. In one embodiment, competitive binding between two antibodies is determined using FACS analysis.

The term “competitive binding assay” is an assay used to determine whether two or more antibodies bind to the same epitope. In one embodiment, a competitive binding assay is a competition fluorescent activated cell sorting (FACS) assay which is used to determine whether two or more antibodies bind to the same epitope by determining whether the fluorescent signal of a labeled antibody is reduced due to the introduction of a non-labeled antibody, where competition for the same epitope will lower the level of fluorescence.

The term “labeled antibody” as used herein, refers to an antibody, or an antigen binding portion thereof, with a label incorporated that provides for the identification of the binding protein, e.g., an antibody. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., 3H, 14C, 35S, 90Y, 99Tc, min, 1251, 1311, 177Lu, 166Ho, or 153Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “CDCP1 associated disorder,” as used herein, includes any disorder or disease (including proliferative disorders, e.g., cancer) that is marked, diagnosed, detected or identified by a phenotypic or genotypic aberration of CDCP1 genetic components or expression during the course or etiology of the disease or disorder. In this regard a CDCP1 phenotypic aberration or determinant may, for example, comprise increased or decreased levels of CDCP1 protein expression on one cell population, e.g., a cancer cell population, as compared to another cell population, e.g., a normal cell population, or increased or decreased CDCP1 protein expression on certain definable cell populations, or increased or decreased CDCP1 protein expression at an inappropriate phase or stage of a cell lifecycle. It will be appreciated that similar expression patterns of genotypic determinants (e.g., mRNA transcription levels) of CDCP1 may also be used to classify or detect CDCP1 associated disorders. In one embodiment, an CDCP1 associated disorder is breast cancer, e.g., triple negative breast cancer. In another embodiment, an CDCP1 associated disorder is colon cancer. In another embodiment, a CDCP1 associated disorder is lung cancer, e.g., non-small cell lung cancer (NSCLC). In another embodiment, an CDCP1 associated disorder is liver cancer. In another embodiment, an CDCP1 associated disorder is pancreatic cancer. In another embodiment, an CDCP1 associated disorder is ovarian cancer. In another embodiment, an CDCP1 associated disorder is kidney cancer.

The term “cancer,” as used herein, is meant to refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include, but are not limited to, breast cancer (Luminal A, TNBC, Ductal), prostate cancer, squamous cell tumors, squamous cell carcinoma (e.g., squamous cell lung cancer or squamous cell head and neck cancer), neuroendocrine tumors, urothelial cancer, vulvar cancer, mesothelioma, liver cancer, bone cancer, pancreatic cancer, skin cancer, cancer or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, environmentally induced cancers including those induced by asbestos, hematologic malignancies including, for example, multiple myeloma, B-cell lymphoma, Hodgkin lymphoma/primary mediastinal B-ceH lymphoma, non-Hodgkin's lymphomas, acute myeloid leukemia (AML), myelodysplastic syndrome (MDS), acute lymphoblastic leukemia (ALL), chronic myelogenous leukemia (CML), myeloproliferative disorders (MPD), chronic lymphoid leukemia, follicular lymphoma, diffuse large B—cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, PVNS, acute myeloid leukemia, adrenocortico carcinoma, ladder urothelial carcinoma, cervical squamous cell carcinoma, endocervical adenocarcinoma, diffuse large B cell lymphoma, glioblastoma multiforme, chronic lymphocytic leukemia, brain lower grade glioma, head and neck squamous cell carcinoma, hepatocellular carcinoma, lung adenocarcinoma, large squamous cell carcinoma, cutaneous melanoma, ovarial serous cystadenocarcinoma, gastric cancer, soft tissue sarcoma, testicular germ cell cancer, thymoma, thyroid carcinoma, uterine corpus endometrial carcinoma, uterine carcinosarcoma, kidney renal clear cell carcinoma, and kidney renal papillary cell carcinoma, and any combinations of said cancers. The present disclosure is also applicable to treatment of metastatic cancers.

In one embodiment, the antibodies of the disclosure are administered to a patient having a solid tumor, including an advanced solid tumor. In one embodiment, the antibodies of the disclosure are administered to a patient having a leukemia. In another embodiment, administration of antibodies of the disclosure induce cell death of CDCP1 expressing cells. The term “CDCP1 expressing tumor,” as used herein, refers to a tumor which expresses CDCP1 protein (including a tumor comprising tumor infiltrating cells that express CDCP1 protein). In one embodiment, CDCP1 expression in a tumor is determined using immunohistochemical staining of tumor cell membranes, where any immunohistochemical staining above background level in a tumor sample indicates that the tumor is a CDCP1 expressing tumor. In another embodiment, a CDCP1 expressing tumor is identified in a patient when greater than 1%, greater than 2%, greater than 3%, greater than 4%, greater than 5%, greater than 6%, greater than 7%, greater than 8%, greater than 9%, greater than 10%, greater than 15%, greater than 20%, greater than 25%, or greater than 30%, greater than 40%, greater than 50%, greater than 60%, greater than 70%, greater than 80%, greater than 90%, or more of the cells in a tumor sample are positive for CDCP1 expression. In another embodiment, CDCP1 positive expression is determined based on membrane staining as determined by, e.g., immunohistochemistry (IHC) analysis.

A CDCP1 expressing tumor is identified as having an “elevated level of CDCP1” or “expressing CDCP1 at an elevated level” when the level of CDCP1 is higher than in tissue surrounding the cancer. In some embodiments, an “elevated level of CDCP1” is one in which 5% or more of the cells in a tumor sample have membrane staining. In some embodiments a “high level” in regard to CDCP1 is 5% or more staining, for example, 5, 10, 20, 30, 40, 50, 60, 70, 80, 90, or 100% of the cells in the tumor sample are stained. In some embodiments, the protein expression levels can be measured by IHC analysis.

A CDCP1 expressing tumor is identified as having a “low level of CDCP1” or “expressing CDCP1 at a low level” is one in which 5% or less of the cells in a tumor sample have membrane staining. In some embodiments a “low level” in regard to CDCP1 is 5% or less staining, for example, 4.9, 4.5, 4, 3, 2, 1, 0.9, 0.8, 0.7, 0.6, 0.5, 0.4, 0.3, 0.2, 0.1% or less of the cells in the tumor sample are stained. In some embodiments, the protein expression levels can be measured by IHC analysis. A cell that expresses no CDCP1 can also be described as expressing a “low level of CDCP1”. Thus, the phrase “expresses a low level of CDCP1” encompasses no CDCP1 expression. In some embodiments, a low level of CDCP1 is within the background staining levels. In some embodiments, a sample that is CDCP1 “negative” has no CDCP1 expression or a low level of CDCP1. In some embodiments, CDCP1 staining is negative when no or less than 5%, 4%, 3%, 2%, or 1% of the cells have membrane staining for CDCP1.

As used herein, the term “tumor sample” refers to a tumor tissue or cell sample obtained from a solid tumor. The sample can include both tumor cells and tumor infiltrating cells, e.g., tumor infiltrating immune cells.

As used herein, the term “non-cancer sample” or “normal sample” refers to a sample from a normal tissue (e.g., a lung or ovarian tissue sample or a normal cell sample). In some embodiments, the non-cancer sample comes from the same subject, but is from a different part of the subject than that being tested. In some embodiments, the non-cancer sample is from a tissue area surrounding or adjacent to the cancer. In some embodiments, the non-cancer sample is not from the subject being tested, but is a sample from a subject known to have, or not to have, a disorder in question (for example, a particular cancer such a CDCP1 related disorder). In some embodiments, the non-cancer sample is from the same subject, but from a point in time before the subject developed cancer. In some embodiments, the reference sample is from a benign cancer sample (for example, benign ovarian cancer sample), from the same or a different subject.

The terms “overexpress,” “overexpression,” or “overexpressed” interchangeably refer to a gene that is transcribed or translated at a detectably greater level, usually in a cancer cell, in comparison to a normal cell. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Thus, overexpression refers to either protein or RNA levels. Overexpression can also be by 5%, 10%, 20%, 30%, 40%, 50%, 60%, 70%, 80%, 90% or more in comparison to a normal cell or comparison cell. In certain embodiments, the anti-CDCP1 antibodies are used to treat solid tumors likely to overexpress CDCP1.

The term “administering” as used herein is meant to refer to the delivery of a substance (e.g., an anti-CDCP1 antibody) to achieve a therapeutic objective (e.g., the treatment of an CDCP1-associated disorder or the inhibition or reduction of a tumor). Modes of administration may be parenteral, enteral and topical. Parenteral administration is usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “combination therapy”, as used herein, refers to the administration of two or more therapeutic substances, e.g., an anti-CDCP1 antibody and an additional therapeutic agent. The additional therapeutic agent may be administered concomitant with, prior to, or following the administration of the anti-CDCP1 antibody. In one embodiment, the anti-CDCP1 antibodies of the present disclosure are administered in combination with one or more immune checkpoint inhibitors or (e.g., one or more antibody or small molecule immune checkpoint inhibitors) for the treatment of a cancer.

As used herein, the term “effective amount” or “therapeutically effective amount” refers to the amount of a drug, e.g., an antibody, which is sufficient to reduce or ameliorate the severity and/or duration of a disorder, e.g., cancer, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of an antibody may, for example, inhibit tumor growth (e.g., inhibit an increase in tumor volume), decrease tumor growth (e.g., decrease tumor volume), reduce the number of cancer cells, and/or relieve to some extent one or more of the symptoms associated with the cancer. The effective amount may, for example, improve disease free survival (DFS), improve overall survival (OS), or decrease likelihood of recurrence.

Various aspects of the present disclosure are described in further detail in the following subsections.

### Anti-CDCP1 Antibodies

In some aspects, the present disclosure provides antibodies or antigen binding fragments thereof that bind to CUB domain-containing protein 1 (CDCP1).

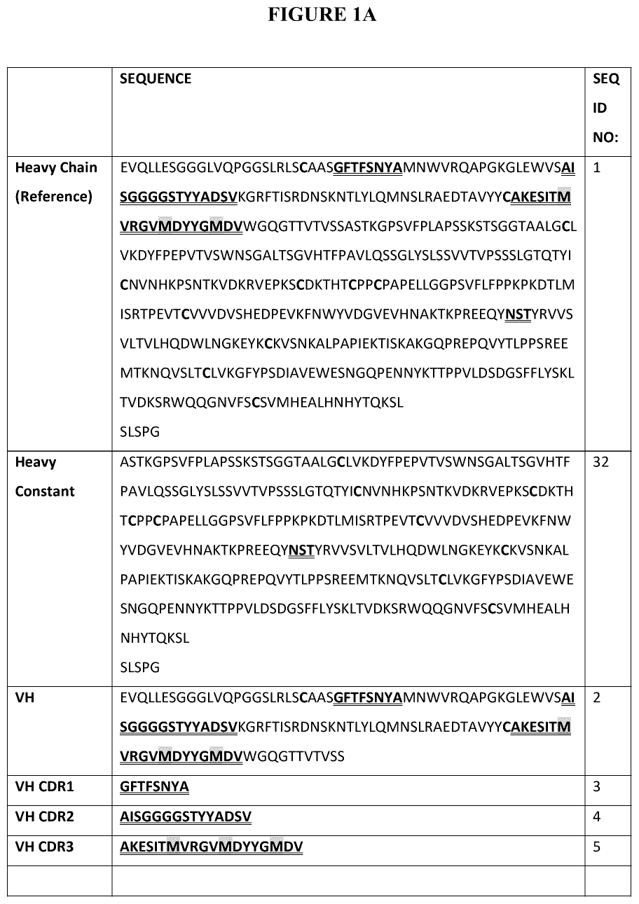

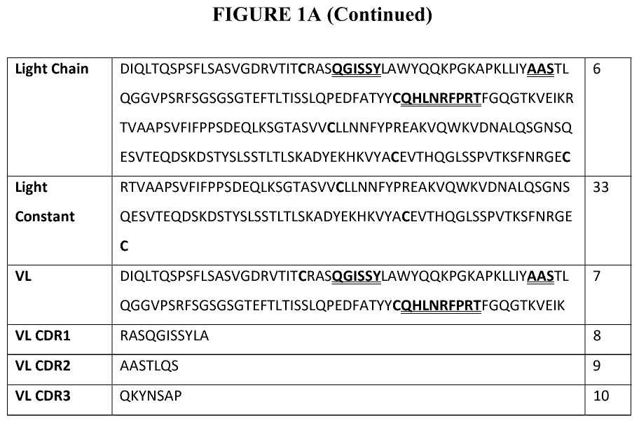

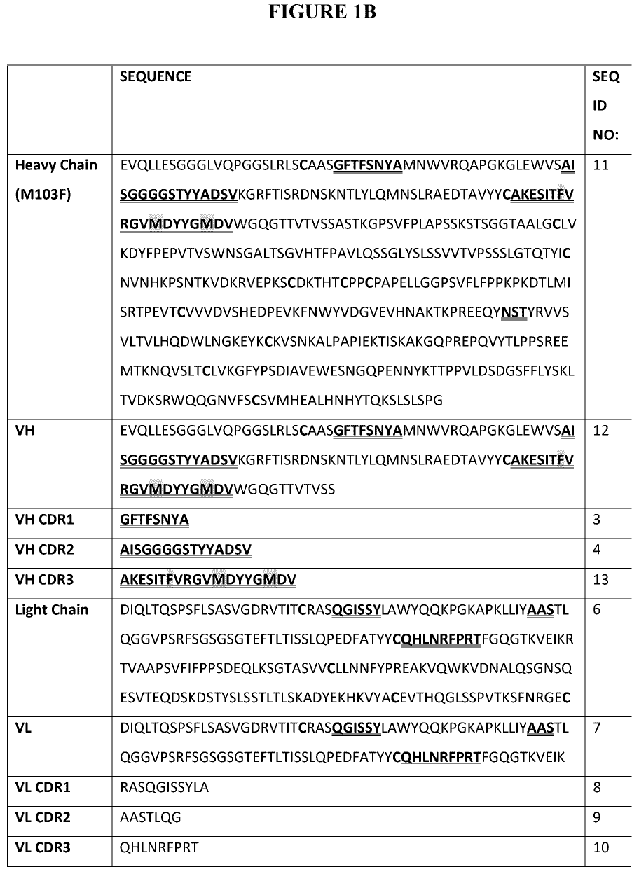

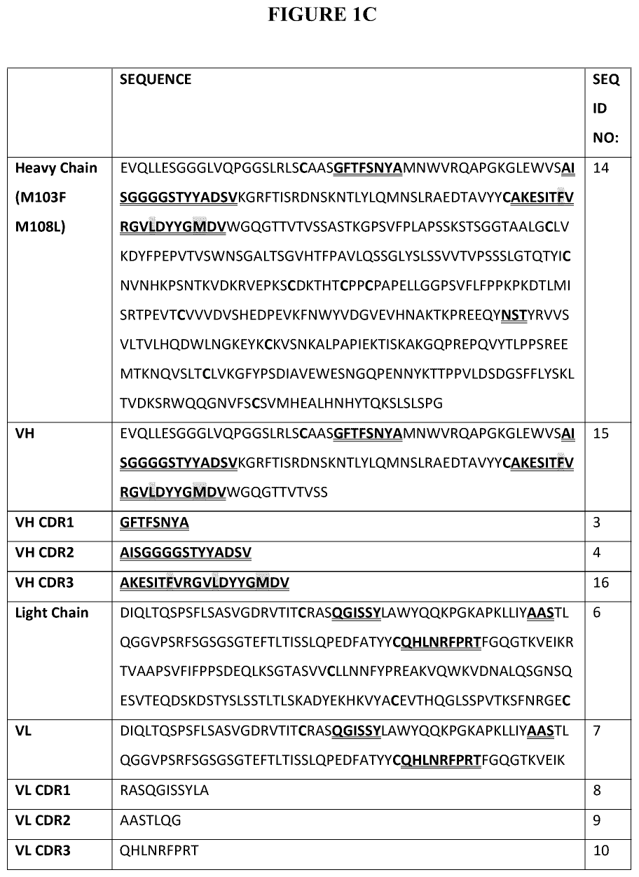

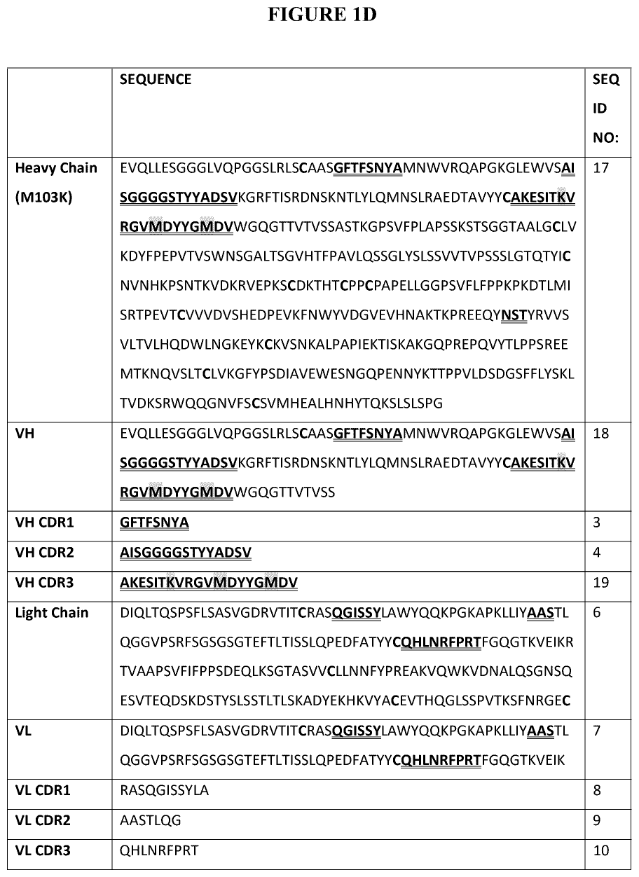

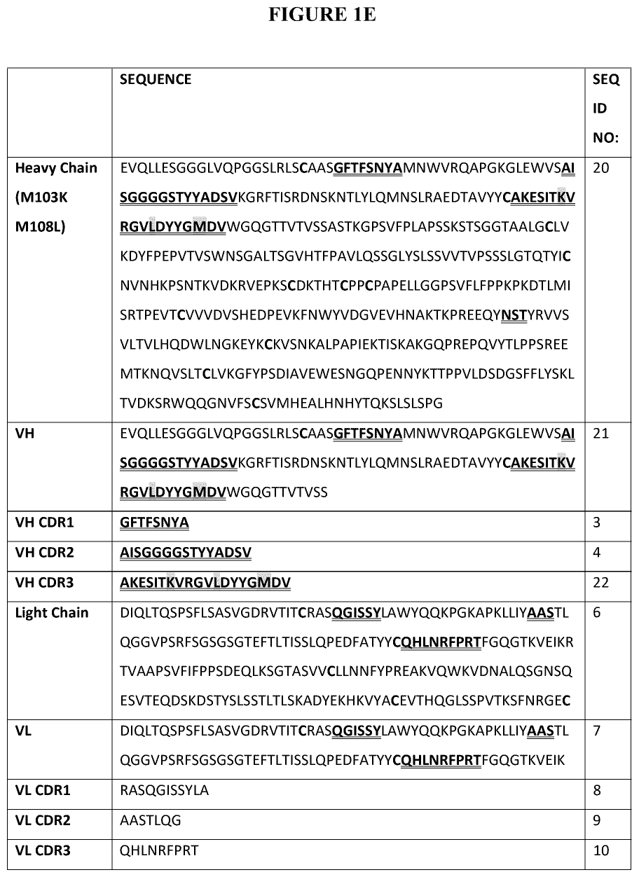

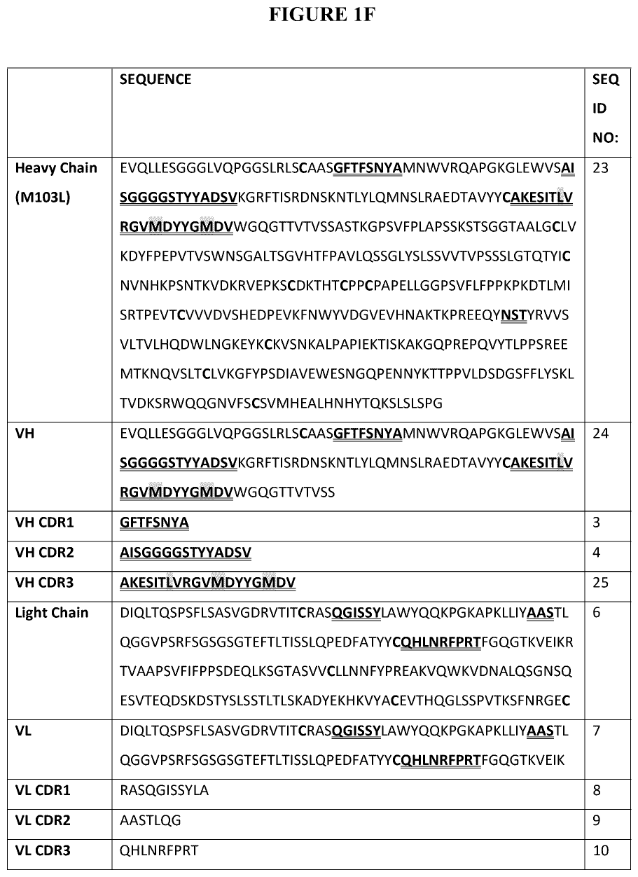

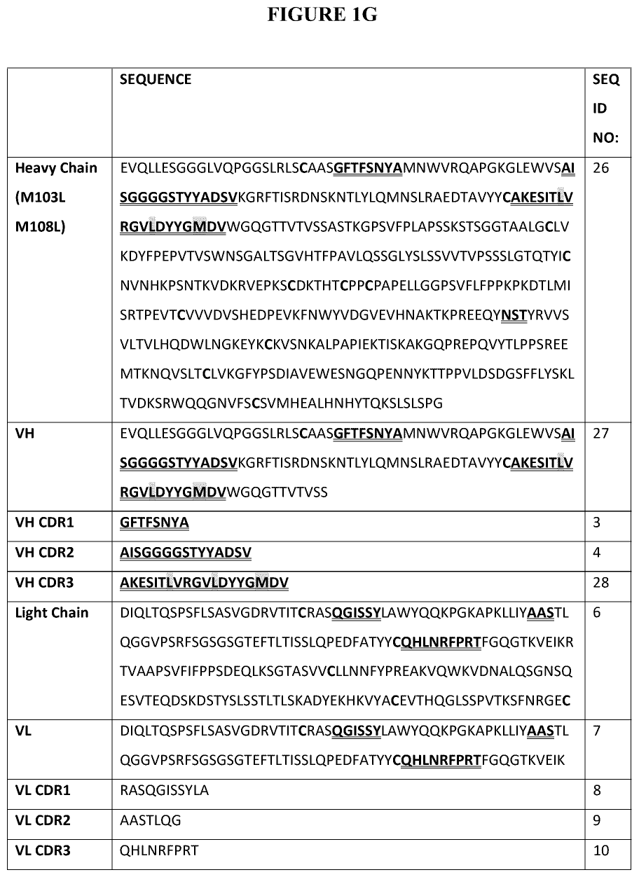

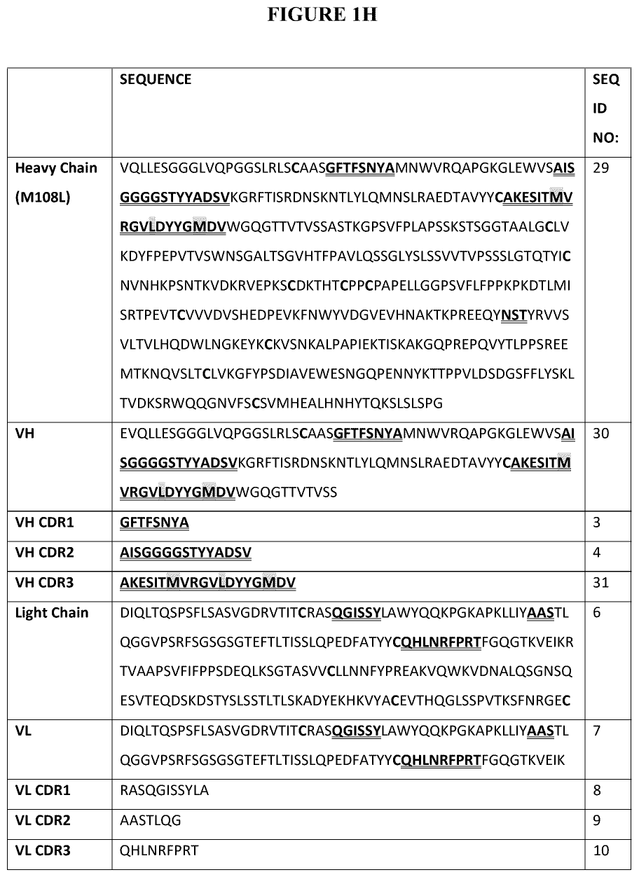

In certain embodiments, the antibodies, or antigen binding fragments thereof, bind to the extracellular domain of CDCP1. In some embodiments, the anti-CDCP1 antibodies and antigen binding fragments thereof comprise one or more heavy chain variable region CDRs and/or one or more light chain variable region CDRs as provided in Tables 1 and 2 (and FIGS. 1A-1H).

In an embodiment, the anti-CDCP1 antibody or binding fragment thereof comprises a VH having a set of CDRs (HCDR1, HCDR2, and HCDR3) disclosed in Table 1. For example, the anti-CDCP1 antibodies or antigen binding fragments thereof may comprise a set of CDRs corresponding to those CDRs disclosed in Table 1.

In another embodiment, the anti-CDCP1 antibodies or antigen binding fragments thereof comprise a VL having a set of CDRs (LCDR1, LCDR2, and LCDR3) as disclosed in Table 2. For example, the anti-CDCP1 antibodies or binding fragment thereof may comprise a set of CDRs corresponding to those CDRs disclosed in Table 2.

TABLE 1
CDR Sequences of Anti-CDCP1 Antibody
Heavy Chains Variable Regions

| Anti-CDCP1 Ab | CDR1 | CDR2 | CDR3 |
|---|---|---|---|
| Reference | SEQ ID NO: 3 | SEQ ID NO: 4 | SEQ ID NO: 5 |
| M103F | SEQ ID NO: 3 | SEQ ID NO: 4 | SEQ ID NO: 13 |
| M103F/M108L | SEQ ID NO: 3 | SEQ ID NO: 4 | SEQ ID NO: 16 |
| M103K | SEQ ID NO: 3 | SEQ ID NO: 4 | SEQ ID NO: 19 |
| M103K/M108L | SEQ ID NO: 3 | SEQ ID NO: 4 | SEQ ID NO: 22 |
| M103L | SEQ ID NO: 3 | SEQ ID NO: 4 | SEQ ID NO: 25 |
| M103L/M108L | SEQ ID NO: 3 | SEQ ID NO: 4 | SEQ ID NO: 28 |
| M108L | SEQ ID NO: 3 | SEQ ID NO: 4 | SEQ ID NO: 31 |

TABLE 2
CDR Sequences of Anti-CDCP1 Light Chains Variable Regions

| Anti-CDCP1 Ab | CDR1 | CDR2 | CDR3 |
|---|---|---|---|
| Reference | SEQ ID NO: 8 | SEQ ID NO: 9 | SEQ ID NO: 10 |
| M103F | SEQ ID NO: 8 | SEQ ID NO: 9 | SEQ ID NO: 10 |
| M103F/M108L | SEQ ID NO: 8 | SEQ ID NO: 9 | SEQ ID NO: 10 |
| M103K | SEQ ID NO: 8 | SEQ ID NO: 9 | SEQ ID NO: 10 |
| M103K/M108L | SEQ ID NO: 8 | SEQ ID NO: 9 | SEQ ID NO: 10 |
| M103L | SEQ ID NO: 8 | SEQ ID NO: 9 | SEQ ID NO: 10 |
| M103L/M108L | SEQ ID NO: 8 | SEQ ID NO: 9 | SEQ ID NO: 10 |
| M108L | SEQ ID NO: 8 | SEQ ID NO: 9 | SEQ ID NO: 10 |

In an alternative embodiment, the anti-CDCP1 antibody or binding fragment thereof comprises a VH having a set of CDRs (HCDR1, HCDR2, and HCDR3) as disclosed in Table 1, and a VL having a set of CDRs (LCDR1, LCDR2, and LCDR3) as disclosed in Table 2.

In some embodiments, the anti-CDCP1 antibody or binding fragment thereof comprises a heavy chain variable (VH) region with one or more amino acids mutations comprising: (a) M103F; (b) M108L; (c) M103K; (d) M103L; (e) M103F and M108L; (f) M103K and M108L; or (g) M103L and M108L (where the amino acid position is based on EU numbering). In one embodiment, the anti-CDCP1 antibody or binding fragment thereof comprises all six of the CDR regions of the M103F, M103F/M108L, M103K, M103K/M108L, M103L, M103L/M108L or M108L antibodies (where the amino acid position is based on EU numbering).

In an embodiment, the anti-CDCP1 antibody or binding fragment thereof comprises a VH having a set of complementarity-determining regions (CDR1, CDR2, and CDR3) selected from the group consisting of:
(i) CDR1: SEQ ID NO: 3, CDR2: SEQ ID NO: 4, CDR3: SEQ ID NO: 13;
(ii) CDR1: SEQ ID NO: 3, CDR2: SEQ ID NO: 4, CDR3: SEQ ID NO: 16;
(iii) CDR1: SEQ ID NO: 3, CDR2: SEQ ID NO: 4, CDR3: SEQ ID NO: 19;
(iv) CDR1: SEQ ID NO: 3, CDR2: SEQ ID NO: 4, CDR3: SEQ ID NO: 22;
(v) CDR1: SEQ ID NO: 3, CDR2: SEQ ID NO: 4, CDR3: SEQ ID NO: 25;
(vi) CDR1: SEQ ID NO: 3, CDR2: SEQ ID NO: 4, CDR3: SEQ ID NO: 28; and
(vii) CDR1: SEQ ID NO: 3, CDR2: SEQ ID NO: 4, CDR3: SEQ D NO: 31.

In an embodiment, the anti-CDCP1 antibody or binding fragment thereof comprises a VL having a set of complementarity-determining regions (CDR1, CDR2, and CDR3) comprising CDR1: SEQ ID NO: 8, CDR2: SEQ ID NO: 9, and CDR3: SEQ ID NO: 10. In another embodiment, the anti-CDCP1 antibodies or antigen binding fragments thereof comprise:
(a) a VH having a set of complementarity-determining regions (CDR1, CDR2, and CDR3) selected from the group consisting of:
   (i) CDR1: SEQ ID NO: 3, CDR2: SEQ ID NO: 4, CDR3: SEQ ID NO: 13;
   (ii) CDR1: SEQ ID NO: 3, CDR2: SEQ ID NO: 4, CDR3: SEQ ID NO: 16;
   (iii) CDR1: SEQ ID NO: 3, CDR2: SEQ ID NO: 4, CDR3: SEQ ID NO: 19;
   (iv) CDR1: SEQ ID NO: 3, CDR2: SEQ ID NO: 4, CDR3: SEQ ID NO: 22;
   (v) CDR1: SEQ ID NO: 3, CDR2: SEQ ID NO: 4, CDR3: SEQ ID NO: 25;
   (vi) CDR1: SEQ ID NO: 3, CDR2: SEQ ID NO: 4, CDR3: SEQ ID NO: 28; and
   (vii) CDR1: SEQ ID NO: 3, CDR2: SEQ ID NO: 4, CDR3: SEQ D NO: 31, and
(b) a VL having a set of complementarity-determining regions (CDR1, CDR2, and CDR3) comprising CDR1: SEQ ID NO: 8, CDR2: SEQ ID NO: 9, and CDR3: SEQ ID NO: 10.

In an embodiment, the CDCP1 antibodies or antigen binding fragments thereof comprise a combination of a VH and a VL having a set of complementarity-determining regions (CDR1, CDR2 and CDR3) selected from the group consisting of:
a) VH: CDR1: SEQ ID NO: 3, CDR2: SEQ ID NO: 4, CDR3: SEQ ID NO: 13, VL: CDR1: SEQ ID NO: 8, CDR2: SEQ ID NO: 9, CDR3: SEQ ID NO: 10;
b) VH: CDR1: SEQ ID NO: 3, CDR2: SEQ ID NO: 4, CDR3: SEQ ID NO: 16, VL: CDR1: SEQ ID NO: 8, CDR2: SEQ ID NO: 9, CDR3: SEQ ID NO: 10;
c) VH: CDR1: SEQ ID NO: 3, CDR2: SEQ ID NO: 4, CDR3: SEQ ID NO: 19, VL: CDR1: SEQ ID NO: 8, CDR2: SEQ ID NO: 9, CDR3: SEQ ID NO: 10;
d) VH: CDR1: SEQ ID NO: 3, CDR2: SEQ ID NO: 4, CDR3: SEQ ID NO: 22, VL: CDR1: SEQ ID NO: 8, CDR2: SEQ ID NO: 9, CDR3: SEQ ID NO: 10;
e) VH: CDR1: SEQ ID NO: 3, CDR2: SEQ ID NO: 4, CDR3: SEQ ID NO: 25, VL: CDR1: SEQ ID NO: 8, CDR2: SEQ ID NO: 9, CDR3: SEQ ID NO: 10;
f) VH: CDR1: SEQ ID NO: 3, CDR2: SEQ ID NO: 4, CDR3: SEQ ID NO: 28, VL: CDR1: SEQ ID NO: 8, CDR2: SEQ ID NO: 9, CDR3: SEQ ID NO: 10; and
g) VH: CDR1: SEQ ID NO: 3, CDR2: SEQ ID NO: 4, CDR3: SEQ ID NO: 31, VL: CDR1: SEQ ID NO: 8, CDR2: SEQ ID NO: 9, CDR3: SEQ ID NO: 10.

In an embodiment, the anti-CDCP1 antibody or binding fragment thereof comprises a heavy chain variable region sequence selected from the group consisting of: SEQ ID NOs: 12, 15, 18, 21, 24, 27 and 30; and/or a light chain variable region sequence consisting of: SEQ ID NO: 7.

In an embodiment, the anti-CDCP1 antibody or binding fragment thereof comprises a pair of heavy chain variable region and light chain variable region sequences, selected from the following combinations: a heavy chain variable region sequence comprising SEQ ID NO: 12 and a light chain variable region sequence comprising SEQ ID NO: 7; a heavy chain variable region sequence comprising SEQ ID NO: 15 and a light chain variable region sequence comprising SEQ ID NO: 7; a heavy chain variable region sequence comprising SEQ ID NO: 18 and a light chain variable region sequence comprising SEQ ID NO: 7; a heavy chain variable region sequence comprising SEQ ID NO: 21 and a light chain variable region sequence comprising SEQ ID NO: 7; a heavy chain variable region sequence comprising SEQ ID NO: 24 and a light chain variable region sequence comprising SEQ ID NO: 7; a heavy chain variable region sequence comprising SEQ ID NO: 27 and a light chain variable region sequence comprising SEQ ID NO: 7; a heavy chain variable region sequence comprising SEQ ID NO: 30 and a light chain variable region sequence comprising SEQ ID NO: 7. The skilled person will further understand that the variable light and heavy chain variable regions may be independently selected, or mixed and matched, to prepare an anti-CDCP1 antibody comprising a combination of variable heavy and light chain variable region that is distinct from the pairings identified above.

In a specific embodiment, the present disclosure provides for antibodies that specifically bind to a CDCP1 antigen, said antibodies comprising the amino acid sequence of one or more of the CDRs in the sequence listing (i.e., SEQ ID NO: 3 for HCDR1; SEQ ID NO: 4 for HCDR2; SEQ ID NOs: 13, 16, 19, 22, 25, 28, or 31 for HCDR3; SEQ ID NO: 8 for LCDR1; SEQ ID NO: 9 for LCDR2; and SEQ ID NO: 3 for LCDR3) and human framework regions with one or more amino acid substitutions at one, two, three or more of the following residues: (a) rare framework residues that differ between the murine antibody framework (i.e., donor antibody framework) and the human antibody framework (i.e., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In certain embodiments, antibodies that specifically bind to a CDCP1 antigen comprising the human framework regions with one or more amino acid substitutions at one, two, three or more of the above-identified residues are antagonistic CDCP1 antibodies.

The present disclosure encompasses antibodies that specifically bind to a CDCP1 antigen, said antibodies comprising the amino acid sequence of the VH domain and/or VL domain in the sequence listing (i.e., SEQ ID NOs: 12, 15, 18, 21, 24, 27, or 30 for VH domains; SEQ ID NO: 7 for VL domains) but having mutations (e.g., one or more amino acid substitutions) in the framework regions. In certain embodiments, antibodies that specifically bind to a CDCP1 antigen comprise the amino acid sequence of the VH domain and/or VL domain or an antigen-binding fragment thereof of an antibody disclosed in the Examples with one or more amino acid residue substitutions in the framework regions of the VH and/or VL domains.

In some embodiments, the anti-CDCP1 antibody or binding fragment thereof that binds to CDCP1 comprises a variable heavy chain sequence that comprises an amino acid sequence with at least about 95%, about 96%, about 97%, about 98%, or about 99%, sequence identity to an amino acid sequence as set forth in any one of SEQ ID NOS: 12, 15, 18, 21, 24, 27 and 30. In other embodiments, the anti-CDCP1 antibody or binding fragment thereof that binds to CDCP1 retains the binding and/or functional activity of a binding module (e.g., antibody) that binds to CDCP1 that comprises the variable heavy chain sequence of SEQ ID NOS: 12, 15, 18, 21, 24, 27 and 30. In still further embodiments, the anti-CDCP1 antibody or binding fragment thereof that binds to CDCP1 comprises the variable heavy chain sequence of SEQ ID NOS: 12, 15, 18, 21, 24, 27 and 30 and has one or more conservative amino acid substitutions, e.g., 1, 2, 3, 4, 5, 1-2, 1-3, 1-4 or 1-5 conservative amino acid substitutions in the heavy chain variable sequence. In yet further embodiments, the one or more conservative amino acid substitutions fall within one or more CDRs and/or framework regions in SEQ ID NOS: 2, 12, 15, 18, 21, 24, 27 and 30 (the amino acid position is based on EU numbering).

In particular embodiments, the anti-CDCP1 antibody or binding fragment thereof that binds to CDCP1 comprises a variable heavy chain sequence with at least about 95%, about 96%, about 97%, about 98%, or about 99% sequence identity to a variable region sequence set forth in any one of SEQ ID NOS: 12, 15, 18, 21, 24, 27 and 30, comprises one or more conservative amino acid substitutions in a framework region (the amino acid position is based on EU numbering), and retains the binding and/or functional activity of a first binding module (e.g., antibody) that binds to CDCP1 and that comprises a variable heavy chain sequence set forth in any one of SEQ ID NOS: 12, 15, 18, 21, 24, 27 and 30 and a variable light chain sequence as set forth in SEQ ID NO: 7.

In some embodiments, the anti-CDCP1 antibody or binding fragment thereof that binds to CDCP1 comprises a variable light chain sequence that comprises an amino acid sequence with at least about 95%, about 96%, about 97%, about 98%, or about 99%, sequence identity to the amino acid sequence set forth in SEQ ID NO: 7. In other embodiments, the anti-CDCP1 antibody or binding fragment thereof that binds to CDCP1 retains the binding and/or functional activity of a binding module (e.g., antibody) that binds to CDCP1 that comprises the variable light chain sequence of SEQ ID NO: 7. In still further embodiments, the anti-CDCP1 antibody or binding fragment thereof that binds to CDCP1 comprises the variable light chain sequence of SEQ ID NO: 7 and has one or more conservative amino acid substitutions, e.g., 1, 2, 3, 4, 5, 1-2, 1-3, 1-4 or 1-5 conservative amino acid substitutions in the light chain variable sequence. In yet further embodiments, the one or more conservative amino acid substitutions fall within one or more CDRs and/or framework regions in SEQ ID NO: 7 (the amino acid position is based on EU numbering).

In an alternative embodiment, the anti-CDCP1 antibody or binding fragment thereof comprises a pair of heavy chain variable region and light chain variable region sequences, selected from the following combinations: a heavy chain variable region sequence that is 90%, 95%, or 99% identical to SEQ ID NO: 12 and a light chain variable region sequence that is 90%, 95%, or 99% identical to SEQ ID NO: 7; a heavy chain variable region sequence that is 90%, 95%, or 99% identical to SEQ ID NO: 15 and a light chain variable region sequence that is 90%, 95%, or 99% identical to SEQ ID NO: 7; a heavy chain variable region sequence that is 90%, 95%, or 99% identical to SEQ ID NO: 18 and a light chain variable region sequence that is 90%, 95%, or 99% identical to SEQ ID NO: 7; a heavy chain variable region sequence that is 90%, 95%, or 99% identical to SEQ ID NO: 21 and a light chain variable region sequence that is 90%, 95%, or 99% identical to SEQ ID NO: 7; a heavy chain variable region sequence that is 90%, 95%, or 99% identical to SEQ ID NO: 24 and a light chain variable region sequence that is 90%, 95%, or 99% identical to SEQ ID NO: 7; a heavy chain variable region sequence that is 90%, 95%, or 99% identical to SEQ ID NO: 27 and a light chain variable region sequence that is 90%, 95%, or 99% identical to SEQ ID NO: 7; a heavy chain variable region sequence that is 90%, 95%, or 99% identical to SEQ ID NO: 30 and a light chain variable region sequence that is 90%, 95%, or 99% identical to SEQ ID NO: 7. The skilled person will further understand that the light and heavy chain variable regions may be independently selected, or mixed and matched, to prepare an anti-CDCP1 antibody comprising a combination of heavy and light chain variable regions that is distinct from the pairings identified above. The binding fragment thereof may comprise at least two, three, four, five, or six CDRs as described herein. The binding fragment further may comprise at least one variable region domain of an antibody described herein. The variable region domain may be of any size or amino acid composition and will generally comprise at least one CDR sequence responsible for binding to human anti-CDCP1, for example, CDR-H1, CDR-H2, CDR-H3, CDR-L1, CDR-L2, and/or CDR-L3 as described herein (also referred to as HCDR1, HCDR2, HCRD3, LCDR1, LCDR2, LCDR3), and which is adjacent to or in frame with one or more framework sequences.

In one aspect, provided herein are humanized anti-CDCP1 antibodies, or antigen binding fragments thereof. In another aspect, provided herein are human anti-CDCP1 antibodies, or antigen binding fragments thereof. In one embodiment, the antibodies or binding fragments thereof bind (e.g., specifically bind) human CDCP1. In another embodiment, the antibodies or antigen binding fragments thereof bind cynomolgus monkey CDCP1. In another embodiment, the antibodies or antigen binding fragments thereof bind (e.g., specifically bind) human CDCP1 expressed on tumor cells.

In some embodiments, the disclosed anti-CDCP1 antibodies specifically bind human CDCP1. In an embodiment, the antibody may be a monoclonal, human, humanized or chimeric antibody, or antigen-binding portions thereof that specifically binds to human CDCP1.

In some embodiments, the anti-CDCP1 antibodies or antigen binding fragments thereof specifically bind to human cells expressing endogenous levels of CDCP1 and to host cells engineered to overexpress CDCP1. The anti-CDCP1 antibodies or antigen binding fragments thereof bind to cells overexpressing human or cyno CDCP1 with subnanomolar EC₅₀ values. In some embodiments, it is advantageous that the disclosed anti-CDCP1 antibodies or antigen binding fragments thereof bind both to hCDCP1 and to cynomolgus monkey CDCP1 (cynoCDCP1). Cross-reactivity with CDCP1 expressed on cells in cynomolgus monkey (e.g. Macaca fascicularis), is advantageous because it enables animal testing of the antibody molecule without having to use a surrogate antibody. The disclosed anti-CDCP1 antibodies all bind to CDCP1 from cynomolgus monkey with notable affinity. These antibodies or antigen binding fragments thereof are characterized by a unique sets of CDR sequences, specificity for CDCP1 and are useful for the treatment of cancer as a monotherapy or in combination with other anti-cancer agents. More specifically, the disclosure relates to antibodies that bind to human CDCP1, and to their use to modulate the CDCP1-mediated activity of cells localized to the tumor microenvironment.

In some embodiments, the anti-CDCP1 antibodies or antigen binding fragments thereof target proteolytic neoepitope on CDCP1.

In some embodiments, the anti-CDCP1 antibodies or antigen binding fragments thereof block CDCP1 and HER2 or EGFR interactions.

In some embodiments, the anti-CDCP1 antibodies or antigen binding fragments thereof exhibit one or more of the following functional characteristics, alone or in combination: (a) is specific for human CDCP1 (b) binds to CUB domain of CDCP1, (c) cross-reacts with cynomolgus CDCP1 (d) disrupts the human CDCP1 binding interaction, (e) targets proteolytic neoepitope on CDCP1, or (f) blocks CDCP1 and HER2 or EGFR interactions; (g) demonstrates ADCC activity to contribute to anti-tumor activity, or (h) enhances the CD8 to Treg ratio within tumors.

In one embodiment, an anti-CDCP1 antibody is the human antibody M103F comprising a heavy chain variable region comprising a CDR1 comprising the amino acid sequence of SEQ ID NO: 3, a CDR2 comprising the amino acid sequence of SEQ ID NO: 4, and a

CDR3 comprising the amino acid sequence of SEQ ID NO: 13, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In some embodiments, disclosed herein is an antibody having a heavy chain variable region comprising the amino acid sequence of SEQ ID NO: 12 and a light chain variable region comprising the amino acid sequence of SEQ ID NO: 7.

In some embodiments, an anti-CDCP1 antibody, or binding fragment thereof, comprises a heavy chain comprising an amino acid sequence set forth in SEQ ID NOs: 1, 11, 14, 17, 20, 23, 26, or 29 or an amino acid sequence having at least 90%, 95%, 96%, 97%, 98%, or 99% identity to SEQ ID NOs: 11, 14, 17, 20, 23, 26, or 29, and/or a light chain comprising an amino acid sequence set forth in SEQ ID NO: 6, or an amino acid sequence having at least 90%, 95%, 96%, 97%, 98%, or 99% identity to SEQ ID NO: 6.

In some embodiments, an anti-CDCP1 antibody, or binding fragment thereof, comprises a heavy chain comprising an amino acid sequence set forth in SEQ ID NO: 11, or an amino acid sequence having at least 90%, 95%, 96%, 97%, 98%, or 99% identity to SEQ ID NO: 11, and/or a light chain comprising an amino acid sequence set forth in SEQ ID NO: 6, or an amino acid sequence having at least 90%, 95%, 96%, 97%, 98%, or 99% identity to SEQ ID NO: 6.

In one embodiment, an anti-CDCP1 antibody is the human antibody M103F/M108L comprising a heavy chain variable region comprising a CDR1 comprising the amino acid sequence of SEQ ID NO: 3, a CDR2 comprising the amino acid sequence of SEQ ID NO: 4, and, a CDR3 comprising the amino acid sequence of SEQ ID NO: 16, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In further embodiments, disclosed herein is an antibody having a heavy chain variable region comprising the amino acid sequence of SEQ ID NO: 15 and a light chain variable region comprising the amino acid sequence of SEQ ID NO: 7.

In some embodiments, an anti-CDCP1 antibody comprises a heavy chain comprising an amino acid sequence set forth in SEQ ID NO: 14, or an amino acid sequence having at least 90%, 95%, 96%, 97%, 98%, or 99% identity to SEQ ID NO: 14, and/or a light chain comprising an amino acid sequence set forth in SEQ ID NO: 6, or an amino acid sequence having at least 90%, 95%, 96%, 97%, 98%, or 99% identity to SEQ ID NO: 6.

In one embodiment, an anti-CDCP1 antibody is the human antibody M103K comprising a heavy chain variable region comprising a CDR1 comprising the amino acid sequence of SEQ ID NO: 3, a CDR2 comprising the amino acid sequence of SEQ ID NO: 4, and a CDR3 comprising the amino acid sequence of SEQ ID NO: 19, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In further embodiments, disclosed herein is an antibody having a heavy chain variable region comprising the amino acid sequence of SEQ ID NO: 18 and a light chain variable region comprising the amino acid sequence of SEQ ID NO: 7.

In some embodiments, an anti-CDCP1 antibody comprises a heavy chain comprising an amino acid sequence set forth in SEQ ID NO: 17, or an amino acid sequence having at least 90%, 95%, 96%, 97%, 98%, or 99% identity to SEQ ID NO: 17, and/or a light chain comprising an amino acid sequence set forth in SEQ ID NO: 6, or an amino acid sequence having at least 90%, 95%, 96%, 97%, 98%, or 99% identity to SEQ ID NO: 6.

In one embodiment, an anti-CDCP1 antibody is the human antibody M103K/M108L comprising a heavy chain variable region comprising a CDR1 comprising the amino acid sequence of SEQ ID NO: 3, a CDR2 comprising the amino acid sequence of SEQ ID NO: 4, and a CDR3 comprising the amino acid sequence of SEQ ID NO: 22, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In further embodiments, disclosed herein is an antibody having a heavy chain variable region comprising the amino acid sequence of SEQ ID NO: 21 and a light chain variable region comprising the amino acid sequence of SEQ ID NO: 7.

In some embodiments, an anti-CDCP1 antibody comprises a heavy chain comprising an amino acid sequence set forth in SEQ ID NO: 20, or an amino acid sequence having at least 90%, 95%, 96%, 97%, 98%, or 99% identity to SEQ ID NO: 20, and/or a light chain comprising an amino acid sequence set forth in SEQ ID NO: 6, or an amino acid sequence having at least 90%, 95%, 96%, 97%, 98%, or 99% identity to SEQ ID NO: 6.

In one embodiment, an anti-CDCP1 antibody is human antibody M103L comprising a heavy chain variable region comprising a CDR1 comprising the amino acid sequence of SEQ ID NO: 3, a CDR2 comprising the amino acid sequence of SEQ ID NO: 4, and a CDR3 comprising the amino acid sequence of SEQ ID NO: 25, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In some embodiments, disclosed herein is an antibody having a heavy chain variable region comprising the amino acid sequence of SEQ ID NO: 24 and a light chain variable region comprising the amino acid sequence of SEQ ID NO: 7.

In some embodiments, an anti-CDCP1 antibody comprises a heavy chain comprising an amino acid sequence set forth in SEQ ID NO: 23, or an amino acid sequence having at least 90%, 95%, 96%, 97%, 98%, or 99% identity to SEQ ID NO: 23, and/or a light chain comprising an amino acid sequence set forth in SEQ ID NO: 6, or an amino acid sequence having at least 90%, 95%, 96%, 97%, 98%, or 99% identity to SEQ ID NO: 6.

In one embodiment, an anti-CDCP1 antibody is the human antibody M103L/M108L comprising a heavy chain variable region comprising a CDR1 comprising the amino acid sequence of SEQ ID NO: 3, a CDR2 comprising the amino acid sequence of SEQ ID NO: 4, and a CDR3 comprising the amino acid sequence of SEQ ID NO: 28, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In further embodiments, disclosed herein is an antibody having a heavy chain variable region comprising the amino acid sequence of SEQ ID NO: 27 and a light chain variable region comprising the amino acid sequence of SEQ ID NO: 7.

In some embodiments, an anti-CDCP1 antibody comprises a heavy chain comprising an amino acid sequence set forth in SEQ ID NO: 26, or an amino acid sequence having at least 90%, 95%, 96%, 97%, 98%, or 99% identity to SEQ ID NO: 26, and/or a light chain comprising an amino acid sequence set forth in SEQ ID NO: 6, or an amino acid sequence having at least 90%, 95%, 96%, 97%, 98%, or 99% identity to SEQ ID NO: 6.

In one embodiment, an anti-CDCP1 antibody is the human antibody M108L comprising a heavy chain comprising a heavy chain variable region comprising a CDR1 comprising the amino acid sequence of SEQ ID NO: 3, a CDR2 comprising the amino acid sequence of SEQ ID NO: 4, and a CDR3 comprising the amino acid sequence of SEQ ID NO: 31, and a light chain variable region comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In further embodiments, disclosed herein is an antibody having a heavy chain variable region comprising the amino acid sequence of SEQ ID NO: 30 and a light chain variable region comprising the amino acid sequence of SEQ ID NO: 7.

In some embodiments, an anti-CDCP1 antibody comprises a heavy chain comprising an amino acid sequence set forth in SEQ ID NO: 29, or an amino acid sequence having at least 90%, 95%, 96%, 97%, 98%, or 99% identity to SEQ ID NO: 29, and/or a light chain comprising an amino acid sequence set forth in SEQ ID NO: 6, or an amino acid sequence having at least 90%, 95%, 96%, 97%, 98%, or 99% identity to SEQ ID NO: 6.

In some embodiments, the disclosed anti-CDCP1 antibodies or antigen binding fragments thereof are human or humanized. In some embodiments, the anti-CDCP1 antibodies or antigen binding fragments thereof specifically bind to the same epitope as the specific antibodies disclosed herein (e.g., anti-CDCP1 variants M103F, M103F/M108L, M103K, M103K/M108L, M103L, M103L/M108L or M108L). Also provided herein are antibodies that specifically bind to specific epitopes on a target CDCP1 molecule.

In another aspect, the present disclosure provides amino acid sequences that comprise or consist of the sequences as shown in Table 6 and FIGS. 1A-1H.

In one aspect, the disclosure provides an antibody or binding fragment thereof that binds to CDCP1 having a heavy chain variable region (VH) including complementarity determining regions (CDRs) 1, 2, 3, wherein the CDR1 region includes an amino acid sequence that is at least 80%, 90%, 92%, 95%, 97%, 98%, 99%, or 100% identical to a selected VH CDR1 amino acid sequence, the CDR2 region includes an amino acid sequence that is at least 80%, 90%, 92%, 95%, 97%, 98%, 99%, or 100% identical to a selected VH CDR2 amino acid sequence, and the CDR3 region includes an amino acid sequence that is at least 80%, 90%, 92%, 95%, 97%, 98%, 99%, or 100% identical to a selected VH CDR3 amino acid sequence, and a light chain variable region (VL) comprising CDRs 1, 2, 3, wherein the CDR1 region comprises an amino acid sequence that is at least 80%, 90%, 92%, 95%, 97%, 98%, 99%, or 100% identical to a selected VL CDR1 amino acid sequence, the CDR2 region comprises an amino acid sequence that is at least 80%, 90%, 92%, 95%, 97%, 98%, 99%, or 100% identical to a selected VL CDR2 amino acid sequence, and the CDR3 region comprises an amino acid sequence that is at least 80%, 90%, 92%, 95%, 97%, 98%, 99%, or 100% identical to a selected VL CDR3 amino acid sequence, wherein the selected VH CDRs 1, 2, 3 amino acid sequences and the selected VL CDRs, 1, 2, 3 amino acid sequences are listed in Table 6 and shown in FIGS. 1A-1H.

In some embodiments, the VH when paired with a VL specifically forms an antigen binding domain that binds to human CDCP1 and/or cynomolgus CDCP1.

In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In another aspect, the CDCP1 antibody or binding fragment thereof comprises a heavy chain variable region (VH) comprising an amino acid sequence that is at least 80%, 90%, 92%, 95%, 97%, 98%, 99%, or 100% identical to a selected VH sequence, and a light chain variable region (VL) comprising an amino acid sequence that is at least 80%, 90%, 92%, 95%, 97%, 98%, 99%, or 100% identical to a selected VL sequence, wherein the selected VH sequence and the selected VL sequence are one of the following: (a) a VH region having an amino acid sequence as set forth in SEQ ID NO: 12 and a VL region having an amino acid sequence as set forth in SEQ ID NO: 7; (b) a VH region having an amino acid sequence as set forth in SEQ ID NOs: 15 and a VL region having an amino acid sequence as set forth in SEQ ID NO: 7; (c) a VH region having an amino acid sequence as set forth in SEQ ID NO: 18 and a VL region having an amino acid sequence as set forth in SEQ ID NO: 7; (d) a VH region having an amino acid sequence as set forth in SEQ ID NO: 21 and a VL region having an amino acid sequence as set forth in SEQ ID NO: 7; (e) a VH region having an amino acid sequence as set forth in SEQ ID NO: 24 and a VL region having an amino acid sequence as set forth in SEQ ID NO: 7; (f) a VH region having an amino acid sequence as set forth in SEQ ID NO: 27 and a VL region having an amino acid sequence as set forth in SEQ ID NO: 7; or, (g) a VH region having an amino acid sequence as set forth in SEQ ID NO: 30 and a VL region having an amino acid sequence as set forth in SEQ ID NO: 7.

In some embodiments, the antibody or binding fragment thereof specifically binds to human CDCP1 and/or cynomolgus CDCP1. In some embodiments, the antibody or binding fragment thereof has a dissociation constant (Kd) for human CDCP1 that is less than 10 nM, and/or a dissociation constant (Kd) for cynomolgus CDCP1 that is less than 10 nM. In some embodiments, the antibody or binding fragment thereof is a humanized antibody or binding fragment thereof.

To generate and to select CDRs having CDCP1 binding and/or neutralizing activity with respect to hCDCP1, standard methods known in the art for generating antibodies, or antigen binding fragments thereof, and assessing the CDCP1 binding and/or neutralizing characteristics of those antibodies or antigen binding fragments thereof, may be used, including but not limited to those specifically described herein.

In some embodiments, the anti-CDCP1 antibodies or antigen binding fragments thereof are part of an antibody drug conjugate (ADC). In some embodiments, various types of linkers known in art such as but not limited to heterobifunctional cross-linkers or charged linkers, can used to conjugate anti-CDCP1 antibodies to drugs to form ADCs. Conjugation of the drug to the antibody via a linker can be accomplished by any technique known in the art. A number of different reactions are available for covalent attachment of drugs and linkers to antibodies. This may be accomplished by reaction of the amino acid residues of the antibody, including the amine groups of lysine, the free carboxylic acid groups of glutamic and aspartic acid, the sulfhydryl groups of cysteine and the various moieties of the aromatic amino acids. The anti-CDCP1 antibodies or antigen binding fragments thereof provide the ADCs with the ability to bind to CDCP1 such that the cytotoxic molecule attached to the antibody or binding fragment thereof may be delivered to the CDCP1-expressing cell, particularly a CDCP1 expressing cancer cell.

The anti-CDCP1 antibodies, or antigen binding fragments thereof, may be able to inhibit or decrease tumor growth in vivo. The tumor can be a CDCP1 negative tumor or an CDCP1 expressing tumor. In various embodiments, anti-CDCP1 antibodies, or antigen binding fragments thereof, are capable of modulating a biological function of CDCP1. In other embodiments of the foregoing aspects, the anti-CDCP1 antibodies, or antigen binding fragments thereof, bind CDCP1 on cells expressing CDCP1. Thus, the disclosure includes anti-CDCP1 antibodies, or antigen binding fragments thereof, that are effective at inhibiting or decreasing tumor growth.

In some embodiments, the anti-CDCP1 antibody or binding fragment thereof comprises one or more conservative amino acid substitutions. A person of skill in the art will recognize that a conservative amino acid substitution is a substitution of one amino acid with another amino acid that has similar structural or chemical properties, such as, for example, a similar side chain. Exemplary conservative substitutions are described in the art, for example, in Watson et al., Molecular Biology of the Gene, The Benjamin/Cummings Publication Company, 4th Ed. (1987).

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., Acta Physiol Scand Suppl 643:55-67, 1998, Sasaki et al., Adv Biophys 35:1-24, 1998). Amino acid substitutions to the antibodies of the disclosure may be made by known methods for example by PCR mutagenesis (U.S. Pat. No. 4,683,195).

In some embodiments, the antibody is a full-length antibody. In other embodiments, the antibody is an antibody fragment including, for example, an antibody fragment selected from the group consisting of: Fab, Fab′, F(ab)₂, Fv, domain antibodies (dAbs), and complementarity determining region (CDR) fragments, single-chain antibodies (scFv), chimeric antibodies, diabodies, triabodies, tetrabodies, miniantibodies, and polypeptides that contain at least a portion of an immunoglobulin that is sufficient to confer CDCP1 specific binding to the polypeptide.

In some embodiments, a variable region domain of an anti-CDCP1 antibody disclosed herein may be covalently attached at a C-terminal amino acid to at least one other antibody domain or a fragment thereof. Thus, for example, a VH domain that is present in the variable region domain may be linked to an immunoglobulin CHI domain, or a fragment thereof. Similarly, a VL domain may be linked to a CK domain or a fragment thereof. In this way, for example, the antibody may be a Fab fragment wherein the antigen binding domain contains associated VH and VL domains covalently linked at their C-termini to a CH1 and CK domain, respectively. The CHI domain may be extended with further amino acids, for example to provide a hinge region or a portion of a hinge region domain as found in a Fab fragment, or to provide further domains, such as antibody CH2 and CH3 domains. In some embodiments, the anti-CDCP1 antibodies or antigen binding fragments thereof may comprise one or both of the antibody constant regions disclosed in SEQ ID NOS: 32 and 33 or a variant thereof. One of skill in the art will also appreciate that in order to evaluate the anti-tumor efficacy of an anti-CDCP1 antibody in a murine tumor model it may be desirable to prepare a recombinant anti-CDCP1 antibody comprising a non-native constant region. In another embodiment, the anti-CDCP1 antibodies or antigen binding fragments thereof may comprise SEQ ID NO: 32 or 33 and have a C- or N-terminal truncation of one or more amino acids (e.g., a C-terminal lysine truncation).

One of skill in the art will recognize that Fc engineering can be used to modify the anti-tumor activities (e.g., effector functions) of the disclosed anti-CDCP1 antibodies or antigen binding fragments thereof to enhance their agonistic activity and/or effector functions. The literature describes several alternative Fc engineering strategies all of which are suitable to design an engineered anti-CDCP1 antibody or binding fragment thereof comprising a variable region of one of the antibodies disclosed herein to modulate CDCP1 expression in either an FcγR dependent or FcγR-independent manner. For example, in order to produce an antibody optimized for immunostimulation, a variable region domain of an anti-CDCP1 antibody disclosed herein may be covalently attached at a C-terminal amino acid to an immunoglobulin Fc domain engineered to confer a low A:I ratio. Therefore, in some embodiments an anti-CDCP1 antibody disclosed herein could be engineered to have enhanced binding to an inhibitory FcγR (e.g., CD32b) in order to stimulate effector T cell activation through hypercrosslinking of CDCP1 trimeric ligand receptor complexes into a supramolecular signaling cluster.

For example, increased CD32b (FcγRIIB) binding affinity can be engineered into a human IgG1 constant region by introducing two mutations S267E and L328F (i.e., “SELF”) (serine at position 267 replaced with glutamic acid and leucine at position 328 replaced with phenylalanine) into a human IgG1 constant region (Chu et al, Mol. Immunol. 45(15):3926-3933, 2008). These two Fc mutations have been reported to increase binding affinity to CD32b by approximately 430-fold, with minimal changes in binding to FcRI and FcRIIA-H131 and eliminating binding to FcRIIIA-V158 compared to WT hIgG1 (Liu et al., Antibodies, 9:64, 2020). In vivo, an S267E/L328F modified anti-CD40 hIgG2 antibody had enhanced ability to activate T cells in hFcR/hCD40 transgenic mice when compared to either WT hIgG1 or hIgG2 variants (Liu et al., Antibodies, 9:64, 2020 and Dahan et al., Cancer Cell, 29, 820-831, 2016). An anti-DR5 antibodies carrying a single S267E (“SE”) mutation has been reported to increase human IgG1 affinity for FcγRIIB several hundred-fold, and to confer improved tumor regression in mouse models humanized for FcγRIIB (Li and Ravetch, Proc. Nat'l Acad. Sci., (USA), 109:10966-71, 2012).

Alternatively, a variable region domain of an anti-CDCP1 antibody or binding fragment thereof may be covalently attached at a C-terminal amino acid to an immunoglobulin Fc domain engineered to comprise either the V12 mutations (E233D/G237D/P238D/H268D/P271G/A330R) or the V11 mutations (G237D/H268D/P271G/A330R) defined by Mimoto et al. The V12 and V11 mutations were elucidated based on studies conducted to expand on the observation that the mutation P238D that enhanced binding to FcγRIIB while either completely abolishing or severely reducing binding to activatory FcRs (FcRI, FcRIIA-H131, FcRIIIA-V131) compared to WT hIgG1 (Mimoto et al., Protein Eng. Des. Sel., 26:589-598, 2013). The V12 and V11 mutations have been reported to enhance FcγRIIB binding approximately 217-fold and 40-fold, respectively, compared to wild type human IgG1 (Mimoto et al.).

In certain embodiments, the antibody or binding fragment thereof comprises a heavy chain constant region, such as an IgG1, IgG2, IgG3, IgG4, IgA, IgE, IgM, or IgD constant region. In certain embodiments, the anti-CDCP1 antibody or binding fragment thereof comprises a heavy chain immunoglobulin constant domain selected from the group consisting of a human IgG constant domain, a human IgM constant domain, a human IgE constant domain, and a human IgA constant domain. In further embodiments, the antibody or binding fragment thereof has an IgG 1 heavy chain constant region, an IgG2 heavy chain constant region, an IgG3 constant region, or an IgG4 heavy chain constant region.

Preferably, the heavy chain constant region is an IgG 1 heavy chain constant region or an IgG4 heavy chain constant region. Furthermore, the antibody or binding fragment thereof can comprise a light chain constant region, either a kappa light chain constant region or a lambda light chain constant region. Preferably, the antibody or binding fragment thereof comprises a kappa light chain constant region. The binding fragment thereof can be, for example, a Fab fragment or a single chain Fv fragment. In certain embodiments, the anti-CDCP1 antibody binding portion is a Fab, a Fab′, a F(ab′)2, a Fv, a disulfide linked Fv, an scFv, a single domain antibody, or a diabody.

In certain embodiments, the anti-CDCP1 antibody is a multispecific antibody, e.g., a bispecific antibody. Replacements of amino acid residues in the Fc portion to alter antibody effector function have been described (Winter, et al. U.S. Pat. Nos. 5,648,260 and 5,624,821, incorporated by reference herein). The Fc portion of an antibody mediates several important effector functions e.g., cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment includes a labeled anti-CDCP1 antibody or binding fragment thereof where the antibody or binding fragment thereof is derivatized or linked to one or more functional molecule(s) (e.g., another peptide or protein). For example, a labeled antibody can be derived by functionally linking an antibody or antibody portion of the disclosure (by chemical coupling, genetic fusion, noncovalent association or otherwise) to one or more other molecular entities, such as another antibody (e.g., a bispecific antibody or a diabody), a detectable agent, a pharmaceutical agent, a protein or peptide that can mediate the association of the antibody or antibody portion with another molecule (such as a streptavidin core region or a polyhistidine tag), and/or a cytotoxic or therapeutic agent selected from the group consisting of a mitotic inhibitor, an antitumor antibiotic, an immunomodulating agent, a vector for gene therapy, an alkylating agent, an antiangiogenic agent, an antimetabolite, a boron-containing agent, a chemoprotective agent, a hormone, an antihormone agent, a corticosteroid, a photoactive therapeutic agent, an oligonucleotide, a radionuclide agent, a topoisomerase inhibitor, a tyrosine kinase inhibitor, a radiosensitizer, and a combination thereof.

Useful detectable agents with which an antibody or binding fragment thereof may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present the addition of hydrogen peroxide and diaminobenzidine leads to a colored reaction product, which is detectable. An antibody may also be derivatized with biotin, and detected through indirect measurement of avidin or streptavidin binding.

In one embodiment, the antibody or binding fragment thereof is conjugated to an imaging agent. Examples of imaging agents that may be used in the compositions and methods described herein include, but are not limited to, a radiolabel (e.g., indium), an enzyme, a fluorescent label, a luminescent label, a bioluminescent label, a magnetic label, and biotin. In one embodiment, the disclosed antibodies or antigen binding fragments thereof are linked to a radiolabel, such as, but not limited to, indium. Indium may be used to label the antibodies described herein for use in identifying CDCP1 positive tumors. In a certain embodiment, the disclosed anti-CDCP1 antibodies are labeled with indium via a bifunctional chelator which is a bifunctional cyclohexyl diethylenetriaminepentaacetic acid (DTPA) chelate (see, U.S. Pat. Nos. 5,124,471; 5,434,287; and 5,286,850, each of which is incorporated herein by reference).

One aspect of the disclosure is directed to generating glycosylation site mutants in which the 0- or N-linked glycosylation site of the anti-CDCP1 antibody or binding fragment thereof has been modified. One skilled in the art can generate such mutants using standard well-known technologies. Glycosylation site mutants that retain the biological activity, but have increased or decreased binding activity, are another object of the disclosure. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in PCT Publication WO 2003/016466A2, and U.S. Pat. Nos. 5,714,350 and 6,350,861, each of which is incorporated herein by reference in its entirety. Additionally or alternatively, a modified anti-CDCP1 antibody or binding fragment thereof can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery.

Cells with altered glycosylation machinery have been described in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP1,176,195; PCT Publications WO 03/035835; WO 99/54342 80, each of which is incorporated herein by reference in its entirety. Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful may include, but are not limited to, glucose, galactose, mannose, fucose, n-acetylglucosamine and sialic acid. Preferably the glycosylated binding protein comprises glycosyl residues such that the glycosylation pattern is human.

### Polynucleotides, Vectors, and Host Cells

In one aspect, isolated nucleic acids are provided. The isolated nucleic acids comprise: a first nucleic acid sequence encoding the heavy chain of the disclosed anti-CDCP1 antibody and a second nucleic acid sequence encoding the light chain of the disclosed anti-CDCP1 antibody. In some embodiments, the first nucleic acid sequence encodes an amino acid sequence as set forth in any one of SEQ ID NOS: 12, 15, 18, 21, 24, and 27. In some embodiments, the second nucleic acid sequence encodes an amino acid sequence as set forth in SEQ ID NO: 7.

Other embodiments encompass isolated polynucleotides that comprise a first nucleic acid sequence encoding an anti-CDCP1 antibody heavy chain or binding fragment thereof and a second nucleic acid sequence encoding an anti-CDCP1 antibody light chain or binding fragment thereof, vectors, and host cells comprising the nucleic acids, and recombinant techniques for production of the antibody or binding fragment thereof. The isolated polynucleotides can encode any desired form of an anti-CDCP1 antibody or binding fragment thereof including, for example, full length monoclonal antibodies, Fab, Fab′ F(ab′)2, and Fv fragments, diabodies, linear antibodies, single-chain antibody molecules, and multi-specific antibodies formed from antibody fragments.

Some embodiments include isolated polynucleotides comprising sequences that encode the heavy chain variable region of an antibody or binding fragment thereof having the amino acid sequence of SEQ ID NOs: 12, 15, 18, 21, 24, 27 and 30. Some embodiments include isolated nucleic acids comprising sequences that encode the light chain variable region of an antibody or binding fragment thereof having the amino acid sequence of SEQ ID NO: 7.

In an embodiment, the isolated polynucleotide sequence(s) encodes an antibody or binding fragment thereof having a light chain and a heavy chain variable region comprising the amino acid sequences of:
(a) a heavy chain variable region sequence comprising SEQ ID NO: 12 and a light chain variable region sequence comprising SEQ ID NO: 7;
(b) a heavy chain variable region sequence comprising SEQ ID NO: 15 and a light chain variable region sequence comprising SEQ ID NO: 7;
(c) a heavy chain variable region sequence comprising SEQ ID NO: 18 and a light chain variable region sequence comprising SEQ ID NO: 7;
(d) a heavy chain variable region sequence comprising SEQ ID NO: 21 and a light chain variable region sequence comprising SEQ ID NO: 7;
(e) a heavy chain variable region sequence comprising SEQ ID NO: 24 and a light chain variable region sequence comprising SEQ ID NO: 7;
(f) a heavy chain variable region sequence comprising SEQ ID NO: 27 and a light chain variable region sequence comprising SEQ ID NO: 7; and
(g) a heavy chain variable region sequence comprising SEQ ID NO: 30 and a light chain variable region sequence comprising SEQ ID NO: 7.

In another embodiment, the isolated polynucleotide sequence(s) encodes an antibody or binding fragment thereof having a heavy chain variable region and a light chain variable region comprising the amino acid sequences of:
(a) a heavy chain variable region sequence that is 90%, 95%, or 99% identical to SEQ ID NO: 12 and a light chain variable region sequence that is 90%, 95%, or 99% identical to SEQ ID NO: 7;
(b) a heavy chain variable region sequence that is 90%, 95%, or 99% identical to SEQ ID NO: 15 and a light chain variable region sequence that is 90%, 95%, or 99% identical to SEQ ID NO: 7;
(c) a heavy chain variable region sequence that is 90%, 95%, or 99% identical to SEQ ID NO: 18 and a light chain variable region sequence that is 90%, 95%, or 99% identical to SEQ ID NO: 7;
(d) a heavy chain variable region sequence that is 90%, 95%, or 99% identical to SEQ ID NO: 21 and a light chain variable region sequence that is 90%, 95%, or 99% identical to SEQ ID NO: 7;
(e) a heavy chain variable region sequence that is 90%, 95%, or 99% identical to SEQ ID NO: 24 and a light chain variable region sequence that is 90%, 95%, or 99% identical to SEQ ID NO: 7;
(f) a heavy chain variable region sequence that is 90%, 95%, or 99% identical to SEQ ID NO: 27 and a light chain variable region sequence that is 90%, 95%, or 99% identical to SEQ ID NO: 7; and
(g) a heavy chain variable region sequence that is 90%, 95%, or 99% identical to SEQ ID NO: 30 and a light chain variable region sequence that is 90%, 95%, or 99% identical to SEQ ID NO: 7.

In another embodiment, the isolated polynucleotide sequence(s) encodes an antibody or binding fragment thereof having a set of heavy chain variable (VH) region CDRs (HCDR1, HCDR2, and HCDR3) disclosed in Table 1 as listed above elsewhere herein.

In yet another embodiment, the isolated polynucleotide sequence(s) encodes an antibody or binding fragment thereof having a set of light chain variable (VL) region CDRs (LCDR1, LCDR2, and LCDR3) disclosed in Table 2 as listed above elsewhere herein. In an alternative embodiment, the isolated polynucleotide sequence(s) encodes an antibody or binding fragment thereof comprising a VH having a set of CDRs (HCDR1, HCDR2, and HCDR3) as disclosed in Table 1, and a VL having a set of CDRs (LCDR1, LCDR2, and LCDR3) as disclosed in Table 2.

The polynucleotide(s) that comprise a sequence encoding an anti-CDCP1 antibody or binding fragment thereof can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

The present disclosure provides a vector(s) (e.g., plasmids) comprising at least one nucleotide sequence encoding at least one of the disclosed anti-CDCP1 antibodies or antigen binding fragments thereof.

In one aspect, provided herein is a vector comprising the disclosed nucleic acids. In some embodiments, the vector is an expression vector. In some embodiments at least two vectors comprise the disclosed nucleic acids. Some embodiments include a first vector comprising a first nucleic acid sequence encoding the heavy chain of the disclosed anti-CDCP1 antibody. Some embodiments include a second vector comprising a second nucleic acid sequence encoding the light chain of the disclosed anti-CDCP1 antibody. In some embodiments, the first vector comprises a first nucleic acid sequence encoding an amino acid sequence as set forth in any one of SEQ ID NOS: 12, 15, 18, 21, 24, and 27. In some embodiments, the second vector comprises a second nucleic acid sequence encoding an amino acid sequence as set forth in SEQ ID NO: 7.

For recombinant production, a polynucleotide encoding the antibody or binding fragment thereof is inserted into a replicable vector for cloning (amplification of the DNA) or for expression. Many suitable vectors for expressing the antibody or binding fragment thereof are available. The vector components generally include, but are not limited to, one or more of the following: a signal sequence, an origin of replication, one or more marker genes, an enhancer element, a promoter, and a transcription termination sequence.

Expression and cloning vectors contain a nucleic acid sequence that enables the vector to replicate in one or more selected host cells. Generally, in cloning vectors such sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2−υ. plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

The present disclosure also provides host cells comprising one or more of the disclosed nucleotide sequences, or one of the disclosed expression vectors.

For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express antibodies or antigen binding fragments thereof in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

In some embodiments, recombinant expression vectors encoding antibody genes are introduced into mammalian host cells, the antibodies or antigen binding fragments thereof are produced by culturing the host cells for a period of time sufficient to allow for expression of the antibody or antigen binding fragments thereof in the host cells or, more preferably, secretion of the antibody or antigen binding fragments thereof into the culture medium in which the host cells are grown.

Non limiting example of mammalian host cells for expressing the antibodies or antigen binding fragments thereof include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mal. Biol. 159:601-621), NSO myeloma cells, COS cells and SP2 cells. In some embodiments, a recombinant expression vector encoding both the antibody heavy chain and the antibody light chain is introduced into CHO cells comprising a glutamine synthase expression system, commercially available from Lonza (hereafter GS-CHO) (Bebbington, C. R. et al. (1992), Biotechnology, 10, pages 169-175).

In some embodiments, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHER gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the disclosure provides a method of synthesizing a recombinant antibody by culturing a host cell in a suitable culture medium until a recombinant antibody is synthesized. Recombinant antibodies may be produced using nucleic acid molecules corresponding to the amino acid sequences disclosed herein. The method can further comprise isolating the recombinant antibody from the culture medium.

### Compositions and Methods of Treatment

The antibodies and antibody portions may be capable of neutralizing human CDCP1 activity both in vivo and in vitro. Accordingly, such antibodies or antigen binding fragments thereof can be used to inhibit CDCP1 activity, e.g., in a cell culture containing CDCP1, in human subjects or in other mammalian subjects having CDCP1 with which an antibody disclosed herein cross-reacts. In one embodiment, the disclosure provides a method for inhibiting CDCP1 activity comprising contacting CDCP1 with an antibody or binding fragment thereof such that CDCP1 activity is inhibited. For example, in a cell culture containing, or suspected of containing CDCP1, an antibody or binding fragment thereof can be added to the culture medium to inhibit CDCP1 activity in the culture. CDCP1 is expressed by numerous solid tumors, including breast cancer tumors, e.g., triple negative breast cancer tumors, lung cancer tumors, e.g., non,-small cell lung cancer tumors, liver cancer tumors, pancreatic cancer tumors, ovarian cancer tumors, kidney cancer tumors, and colon cancer tumors. Accordingly, the anti-CDCP1 antibodies, and antigen binding fragments thereof, can be used for the treatment of breast cancer, e.g., triple negative breast cancer, lung cancer, e.g., non,-small cell lung cancer, liver cancer, pancreatic cancer, ovarian cancer, kidney cancer, and colon cancer in a subject. In one embodiment, greater than 1%, greater than 2%, greater than 3%, greater than 4%, greater than 5%, greater than 6%, greater than 7%, greater than 8%, greater than 9%, greater than 10%, greater than 15%, greater than 20%, greater than 25%, or greater than 30%, greater than 40%, greater than 50%, greater than 60%, greater than 70%, greater than 80%, greater than 90%, or more of the cells in a tumor sample, e.g., breast cancer tumor, e.g., triple negative breast cancer tumor, lung cancer tumor, e.g., non,-small cell lung cancer tumor, liver cancer tumor, pancreatic cancer tumor, ovarian cancer tumor, kidney cancer tumor, and colon cancer tumor, are positive for CDCP1 expression.

In another embodiment, a tumor sample has a high level of CDCP1 expression. For example, in one embodiment, at least 5% or more of the cells in a tumor sample. e.g., breast cancer tumor, e.g., triple negative breast cancer tumor, lung cancer tumor, e.g., non,-small cell lung cancer tumor, liver cancer tumor, pancreatic cancer tumor, ovarian cancer tumor, kidney cancer tumor, and colon cancer tumor, have membrane staining. In another embodiment, a tumor sample obtained from the subject displays a low level of expression of CDCP1. The expression level of CDCP1 can be determined by any method known in the art. For example, the expression level of CDCP1 can be determined via immunohistochemical analysis. In another embodiment, the cancer has been previously treated with another anti-cancer agent or anti-cancer therapy, e.g., a chemotherapy. In one embodiment, the cancer is resistant to chemotherapy.

In another embodiment, disclosed herein is a method for reducing CDCP1 activity in a subject, advantageously from a subject suffering from a CDCP1 associated disorder, e.g., cancer such as breast cancer, lung cancer, small cell lung cancer, liver cancer, pancreatic cancer, ovarian cancer, kidney cancer, and colon cancer, or a disorder in which CDCP1 activity is detrimental. The disclosure provides methods for reducing CDCP1 activity in a subject suffering from such a disease or disorder, which method comprises administering to the subject an antibody or binding fragment thereof such that CDCP1 activity in the subject is reduced. Preferably, the CDCP1 is human CDCP1, and the subject is a human subject. Alternatively, the subject can be a mammal expressing a CDCP1 to which antibodies of the disclosure are capable of binding. Still further the subject can be a mammal into which CDCP1 has been introduced (e.g., by administration of CDCP1 or by expression of a CDCP1 transgene).

The antibodies or antigen binding fragments thereof can be administered to a human subject for therapeutic purposes. Moreover, the antibodies or antigen binding fragments thereof can be administered to a non-human mammal expressing a CDCP1 with which the antibody is capable of binding for veterinary purposes or as an animal model of human disease. Regarding the latter, such animal models may be useful for evaluating the therapeutic efficacy of antibodies of the disclosure (e.g., testing of dosages and time courses of administration). The disclosure also provides compositions including, for example, pharmaceutical compositions that comprise an anti-CDCP1 antibody or binding fragment thereof for use as a therapeutic drug for the treatment of patients having an epithelial cell-derived primary or metastatic cancer. In a particular embodiment, a therapeutically effective amount of the compositions described herein are administered to cancer patients to kill tumor cells. For example, the compositions described herein can be used to treat a patient with a tumor characterized by the presence of cancer cells expressing or overexpressing CDCP1.

A tumor may be a solid tumor or a liquid tumor. In certain embodiments, a tumor is an immunogenic tumor. In certain embodiments, a tumor is non-immunogenic. Non-limiting examples of cancers for treatment include squamous cell carcinoma, small-cell lung cancer, non-small cell lung cancer, glioma, gastric cancer, renal cancer, ovarian cancer, liver cancer, colorectal cancer, kidney cancer, prostate cancer, thyroid cancer, neuroblastoma, pancreatic cancer, breast cancer, head and neck cancer, melanoma, bone cancer, uterine cancer, and other hematologic malignancies derived from either of the two major blood cell lineages such as myeloid cell line of lymphoid cell line.

In some aspects, the treatment of cancer represents a field where combination strategies are especially desirable since frequently the combined action of two, three, four or even more cancer drugs/therapies generates synergistic effects which are considerably stronger than the impact of a mono-therapeutic approach. The agents and compositions (e.g., pharmaceutical compositions) provided herein may be used alone or in combination with conventional therapeutic regimens such as surgery, irradiation, chemotherapy and/or bone marrow transplantation (autologous, syngeneic, allogeneic or unrelated). The agents and compositions may also be used in combination with one or more of an antineoplastic agent, a chemotherapeutic agent, a growth inhibitory agent, a cytotoxic agent, an immune checkpoint inhibitor, costimulatory molecule, kinase inhibitors, angiogenesis inhibitors, small molecule targeted therapy drugs, and multi-epitope strategies. Thus, in another embodiment of the present disclosure, a cancer treatment may be effectively combined with various other drugs.

The disclosed anti-CDCP1 antibodies or antigen binding fragments thereof can be administered either alone or in combination with other compositions that are useful for treating cancer. In one embodiment, the disclosed antibodies or antigen binding fragments thereof can be administered either alone or in combination with other immunotherapeutics including other antibodies useful for treating cancer. For example, in an embodiment the other immunotherapeutic is an antibody against an immune checkpoint molecule selected from the group consisting of human programmed cell death protein 1 (PD-1), PD-L1 and PD-L2, lymphocyte activation gene 3 (LAG3), NKG2A, B7-H3, B7-H4, CTLA-4, GITR, VISTA, CD137, TIGIT and any combination thereof. In an alternative embodiment the second immunotherapeutic is an antibody to a tumor specific antigen (TSA) or a tumor associated antigen (TAA). Each combination representing a separate embodiment of the disclosure.

An anti-CDCP1 antibody or binding fragment thereof may be able to be combined with an immunogenic agent (tumor vaccines) such as cancer cells, purified tumor antigen including recombinant proteins, peptides and carbohydrate molecules.

An anti-CDCP1 antibody or binding fragment thereof may be combined with checkpoint inhibitors such as PD1/PDL1 blockers, and other therapies that can overcome the tumor immune escape, such as PDL1/TGFb trap. Targeting CDCP1 synergizes with anti-PD-1 in animal models (Wei et al., AACR 2020, Poster #2282) indicating that CDCP1 costimulation and PD1 blockade could lead to an enhanced anti-tumor immune response than PD1 monotherapy.

The disclosed anti-CDCP1 antibodies or antigen binding fragments thereof can be combined with standard cancer treatment (e.g. surgery, radiation and chemotherapy). In these cases, it may be possible to reduce the dose of chemotherapy, improve the efficacy of chemotherapy and radiation therapy in cancer patients and prolong their survival. In another aspect, the present disclosure provides a method for treating cancer, comprising administering a therapeutically effective amount of an antibody or binding fragment thereof as described herein, to a subject in need thereof.

In some embodiments, the cancer is selected from the group consisting of breast cancer, lung cancer, small cell lung cancer, liver cancer, pancreatic cancer, ovarian cancer, kidney cancer, and colon cancer. In one embodiment, the cancer is breast cancer, e.g., triple negative breast cancer. In one embodiment, the cancer is colon cancer. In one embodiment, the cancer is lung cancer, e.g., non-small cell lung cancer (NSCLC).

In another aspect, the present disclosure provides a method for inhibiting or decreasing solid tumor growth in a subject having a solid tumor. The method includes administering a therapeutically effective amount of an antibody or binding fragment thereof, as described herein, to the subject having the solid tumor, such that the solid tumor growth is inhibited or decreased.

In one embodiment, the antibody or binding fragment thereof as described herein is administered in combination with an additional agent or an additional therapy. In one embodiment, the additional agent is an immune checkpoint inhibitor, e.g., an antibody, such as an antibody selected from the group consisting of an anti-PD1 antibody, an anti-PDL1 antibody and an anti-CTLA-4 antibody. In one embodiment, the additional therapy is radiation. In one embodiment, the additional agent is a chemotherapeutic agent. In another aspect of the present disclosure, the present disclosure provides a method for treating cancer. The method includes administering a therapeutically effective amount of an anti-CDCP1 antibody or binding fragment thereof, as described herein, and an anti-PDL1 antibody to a subject in need thereof. In one embodiment, the cancer or tumor is characterized as having CDCP1 phosphorylated or overexpressed.

In yet another aspect, the disclosure features methods of inhibiting or decreasing proliferation of a cell (e.g., a cell that overexpresses CDCP1, or a cell characterized by the presence of CDCP1 on the cell surface) that include contacting the cell with an antibody or binding fragment thereof disclosed herein. In another aspect, the disclosure features methods of inhibiting or decreasing proliferation of a cancer cell (e.g., a cancer cell that overexpresses CDCP1, or a cancer cell characterized by having CDCP1 on the cell surface) that include contacting the cancer cell with an antibody or binding fragment thereof disclosed herein. A cell can be identified as overexpressing CDCP1 protein using any of the methods described herein. In some embodiments, a cell can also be identified as having CDCP1 on the cell surface using any of the methods described herein. In some implementations, the cell death induced by the antibody is complement-dependent cytotoxicity and/or cell-dependent cell cytotoxicity. In some implementations, the antibody is conjugated to a therapeutic agent (e.g., a cytotoxic agent) and the cell death is induced by the endocytosis of the antibody into the cancer cells, which in turn triggers the death of the cancer cells. In some implementations, the methods include the step of administering to a subject (e.g., a subject in need thereof) an effective amount of an antibody or binding fragment thereof disclosed herein, thereby selectively inducing cell death.

Methods for identifying CDCP1 expressing tumors are known in the art, and include FDA-approved tests and validation assays. For example, these assays may use primers that are specific for the CDCP1 gene and/or cDNA and result in the amplification of the CDCP1 gene/cDNA, or a portion thereof. The amplified PCR products may be subsequently analyzed, for example, by gel electrophoresis using standard methods known in the art to determine the size of the PCR products. Such tests may be used to identify tumors that may be treated with the methods and compositions described herein.

In another aspect, this application features a method of treating (e.g., curing, suppressing, ameliorating, delaying or preventing the onset of, or preventing recurrence or relapse of) or preventing a CDCP1-associated disorder, in a subject. The method includes: administering to the subject a CDCP1 binding agent (particularly an antagonist), e.g., an anti-CDCP1 antibody or fragment thereof as described herein, in an amount sufficient to treat or prevent the CDCP1-associated disorder. The CDCP1 antagonist, e.g., the anti-CDCP1 antibody or fragment thereof, can be administered to the subject, alone or in combination with other therapeutic modalities as described herein.

Antibodies or antigen binding fragments thereof, can be used alone or in combination with an additional agent to treat such diseases. In some embodiments, the additional agent can be a therapeutic agent art-recognized as being useful to treat the disease or condition being treated by the antibody. The additional agent also can be an agent that imparts a beneficial attribute to the therapeutic composition, e.g., an agent which affects the viscosity of the composition.

It should further be understood that the combinations which are to be included within this disclosure are those combinations useful for their intended purpose. The agents set forth below are illustrative for purposes and not intended to be limited. The combinations, which are part of this disclosure, can be the antibodies of the disclosure and at least one additional agent selected from the lists provided below herein. The combination can also include more than one additional agent, e.g., two or three additional agents if the combination is such that the formed composition can perform its intended function.

The combination therapy can include one or more CDCP1 antagonists, e.g., anti-CDCP1 antibodies or fragments thereof, formulated with, and/or co-administered with, one or more additional therapeutic agents, e.g., one or more cytokine and growth factor inhibitors, immunosuppressants, anti-inflammatory agents (e.g., systemic anti-inflammatory agents), anti-fibrotic agents, metabolic inhibitors, enzyme inhibitors, and/or cytotoxic or cytostatic agents, mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, or radiosensitizers, as described in more herein.

In a particular embodiment, the anti-CDCP1 antibodies disclosed herein are used in combination with an anti-cancer agent or an antineoplastic agent. The terms “anti-cancer agent” and “antineoplastic agent” refer to drugs used to treat malignancies, such as cancerous growths. In one embodiment, the disclosed anti-CDCP1 antibodies are administered in combination with one or more immune checkpoint inhibitors (e.g., antibody or small molecule immune checkpoint inhibitors) for the treatment of a cancer. In some embodiments, the immune checkpoint inhibitor is an inhibitor of Programmed Death-Ligand 1 (PDL1, also known as B7-HI, CD274), Programmed Death 1 (PD-1), CTLA-4, PD-L2 (B7-DC, CD273), LAG3, TIM3, 2B4, A2aR, B7HI, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDOI, IDO2, ICOS (inducible T cell costimulator), KIR, LAIRI, LIGHT, MARCO (macrophage receptor with collageneous structure), PS (phosphatidylserine), OX-40, SLAM, TIGHT, VISTA, or any combinations thereof. In some embodiments, the immune checkpoint inhibitor is an anti-CTLA-4, PD-LI, or PD-I antibody therapy such as, but not limited to Yervoy® (ipilimumab Bristol-Myers Squibb), Opdivo® (nivolumab; Bristol-Myers Squibb), Keytruda® (pembrolizumab; Merck), and Tecentriq® (atezolizumab; Roche).

In some embodiments, the anti-CDCP1 antibodies disclosed herein are used in combination with an inhibitor of the PI3K-AKT mTOR pathway, e.g., rapamycin, temsirolimus, everolimus, ridaforolimus, Torinl, Torin2, PP242, KU63794, WYE354, NVP-BEZ235, XL765, GDC-0491, GDC-0980, GSK2126458, AZD8055, OSI-027, CH5132799, PF-05212384, or ZSTK474. See also WO 2012/054748; U.S. Pat. No. 8,394,818; McCubrey et al., 2012, Oncotarget, 3:1068-1111.

In one embodiment, the disclosed anti-CDCP1 antibodies are administered in combination with a PARP (poly ADP ribose polymerase) inhibitor. PARP inhibitors are well known to those of ordinary skill in the art and include, but are not limited to, Niraparib, Olaparib, Rucaparib, Iniparib, Talazoparib, Veliparib, CEP 9722, E7016, BGB-290, and 3-aminobenazamine.

Drug therapy may be used alone, or in combination with other treatments such as surgery or radiation therapy. Several classes of drugs may be used in cancer treatment, depending on the nature of the organ involved. For example, breast cancers are commonly stimulated by estrogens, and may be treated with drugs which inactive the sex hormones. Similarly, prostate cancer may be treated with drugs that inactivate androgens, the male sex hormone. In some embodiments, the anti-CDCP1 antibodies can be administered alone or with another anti-cancer agent which acts in conjunction with or synergistically with the antibody to treat the disease associated with CDCP1 activity. Such anti-cancer agents include, for example, agents well known in the art (e.g., cytotoxins, chemotherapeutic agents, small molecules and radiation). Examples of anti-cancer agents include, but are not limited to, Panorex (Glaxo-Welcome), Rituxan (IDEC/Genentech/Hoffman la Roche), Mylotarg (Wyeth), Campath (Millennium), Zevalin (IDEC and Schering AG), Bexxar (Corixa/GSK), Erbitux (Imclone/BMS), Avastin (Genentech) and Herceptin (Genentech/Hoffman la Roche). Other anti-cancer agents include, but are not limited to, those disclosed in U.S. Pat. No. 7,598,028 and International Publication No. WO2008/100624, the contents of which are hereby incorporated by reference. One or more anti-cancer agents may be administered either simultaneously or before or after administration of an antibody or antigen binding portion thereof.

In some embodiments, the anti-CDCP1 antibodies described herein can be used in a combination therapy with an inhibitor of NAMPT (see examples of inhibitors in US 2013/0303509; AbbVie, Inc., incorporated by reference herein) to treat a subject in need thereof. NAMPT (also known as pre-B-cell-colony-enhancing factor (PBEF) and visfatin) is an enzyme that catalyzes the phosphoribosylation of nicotinamide and is the rate-limiting enzyme in one of two pathways that salvage NAD. In one embodiment, the anti-CDCP1 antibodies described herein are administered in combination with a NAMPT inhibitor for the treatment of cancer in a subject. In some embodiments, the anti-CDCP1 antibodies described herein can be used in a combination therapy with SN-38, which is the active metabolite of the topoisomerase inhibitor irinotecan.

In another aspect, this application provides a method for detecting the presence of CDCP1 in a sample in vitro (e.g., a biological sample, such as serum, plasma, tissue, biopsy). The subject method can be used to diagnose a disorder, e.g., a cancer. The method includes: (i) contacting the sample or a control sample with the anti-CDCP1 antibody or fragment thereof as described herein; and (ii) detecting formation of a complex between the anti-CDCP1 antibody or fragment thereof, and the sample or the control sample, wherein a statistically significant change in the formation of the complex in the sample relative to the control sample is indicative of the presence of CDCP1 in the sample. Given their ability to bind to human CDCP1, the anti-human CDCP1 antibodies, or portions thereof, can be used to detect human CDCP1 (e.g., in a biological sample, such as serum or plasma), using a conventional immunoassay, such as an enzyme linked immunosorbent assays (ELISA), an radioimmunoassay (RIA) or tissue immunohistochemistry. In one aspect, the disclosure provides a method for detecting human CDCP1 in a biological sample comprising contacting a biological sample with an antibody, or antibody portion, and detecting either the antibody (or antibody portion) bound to human CDCP1 or unbound antibody (or antibody portion), to thereby detect human CDCP1 in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include 3H, 14C, 35S, 90Y, 99Tc, 111In, 12s1, 131I, 177Lu, 166Ho, or 1s3 Sm.

Alternative to labeling the antibody, human CDCP1 can be assayed in biological fluids by a competition immunoassay utilizing rhCDCP1 standards labeled with a detectable substance and an unlabeled anti-human CDCP1 antibody. In this assay, the biological sample, the labeled rhCDCP1 standards and the anti-human CDCP1 antibody are combined and the amount of labeled rhCDCP1 standard bound to the unlabeled antibody is determined. The amount of human CDCP1 in the biological sample is inversely proportional to the amount of labeled rhCDCP1 standard bound to the anti-CDCP1 antibody. Similarly, human CDCP1 can also be assayed in biological fluids by a competition immunoassay utilizing rhCDCP1 standards labeled with a detectable substance and an unlabeled anti-human CDCP1 antibody.

In yet another aspect, this application provides a method for detecting the presence of CDCP1 in vivo (e.g., in vivo imaging in a subject). The subject method can be used to diagnose a disorder, e.g., a CDCP1-associated disorder. The method includes: (i) administering the anti-CDCP1 antibody or fragment thereof as described herein to a subject or a control subject under conditions that allow binding of the antibody or fragment to CDCP1; and (ii) detecting formation of a complex between the antibody or fragment and CDCP1, wherein a statistically significant change in the formation of the complex in the subject relative to the control subject is indicative of the presence of CDCP1.

### Diagnostics and Imaging

The antibodies or antigen binding fragments thereof of the present disclosure can also be used in various diagnostic, and imaging methods. For example, the antibodies of the disclosure can be used in any known assay method, such competitive binding assays, direct and indirect sandwich assays, and immunoprecipitation assays (see, e.g., Zola, Monoclonal Antibodies: A Manual of Techniques, pp. 147-158 (CRC Press, Inc., 1987). For use in such methods, e.g., for use in in vitro assays, the antibodies can be detectably labeled, e.g., with a fluorophore such as Fluorescein isothiocyanate (FITC) or phycoerythrin or with an enzyme substrate, such as a substrate for horse radish peroxidase, for easy detection. As discussed herein, the antibodies antigen binding fragments thereof of the disclosure also can be used for in vivo diagnostic assays and in vivo imaging. In some implementations, the antibody is labeled with a radionucleotide (such as 3H, min, 14C, 32P, or 1231) so that the cells or tissue of interest can be localized and/or imaged using immunoscintigraphy. Methods of conjugating labels to an antibody are known in the art. In other implementations of the disclosure, antibodies disclosed herein need not be labeled, and the presence thereof can be detected using a labeled antibody, which binds to the antibody. For example, the disclosure provides methods of detecting a CDCP1 protein in a sample (e.g., a sample containing mammalian cells, e.g., a biopsy sample) that include contacting a sample with an antibody disclosed herein and detecting binding of the antibody to any CDCP1 protein in the sample. Some implementations further include recording the detection or non-detection of CDCP1 protein (e.g., the presence, the detection, the non-detection, and/or level of a CDCP1 protein) in the clinical records of a subject from whom the sample was obtained.

In some implementations, the clinical record is stored on a computer-readable medium, e.g., a disc, tape, or computer memory. Some implementations further include administering any one of the antibodies described herein to a subject identified as having detectable CDCP1 protein or an elevated level of a CDCP1 protein (e.g., as compared to a reference level, e.g., a level of a CDCP1 protein produced by a non-cancerous cell) in his or her sample. Some implementations further include performing further testing for the presence of cancer (e.g., any of the methods for further testing for the presence of cancer described herein) on a subject identified as having detectable CDCP1 protein or an elevated level of a CDCP1 protein. Additional examples of reference values are described herein. Also provided are methods of imaging one or more cancer cells (e.g., a cancer cell that overexpresses CDCP1 or a cancer cell characterized by having CDCP1 on the cell surface, e.g., breast cancer (e.g., triple-negative breast cancer), carcinoid cancer, cervical cancer, endometrial cancer, glioma, head and neck cancer, liver cancer, lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy cell) in a subject (e.g., a subject in need thereof, e.g., a subject identified as being at risk for developing a cancer, a subject suspected of having a cancer, or a subject already diagnosed or identified as having a cancer), that include administering to a subject an antibody that is conjugated to a detectable label (e.g., any of the examples of detectable labels described herein) and imaging the presence of the cancer cell by detecting the detectable label in the subject. In some implementations, the detectable label is a fluorophore, a metalloporphyrin, a paramagnetic metal, a superparamagnetic metal, a magnetic particle (e.g., 10-20 nm in diameter), a nitroxide stable free radical, or ferrioxamine methane sulfonate, or a metal, e.g., gold.

The methods of detection and diagnosis can be performed on any biological sample (e.g., a sample containing mammalian cells), e.g., a sample from an individual having a cancer or suspected of having a cancer. The biological sample can be, e.g., a biopsy (e.g., needle biopsy), tissue section, or a bodily fluid (e.g., lung gavage, urine, saliva, blood, tears, semen, or breast milk).

The antibody can also be used as staining reagent in pathology, following techniques that are well known in the art. Such detection of specific binding by the antibody to the sample (e.g., detection of an antibody: sample complex) can be made by any known method including, without limitation, western blotting analysis, immunohistochemistry (IHC) analysis, immunofluorescence (IF) analysis, flow cytometry analysis, FACS analysis, ELISA, and immunoprecipitation. See, generally, Immunological Methods, Vols. I and II (Lefkovits and Pernis, eds., Academic Press, NY, 1979 and 1981, herein incorporated by reference. Cellular extracts of the foregoing biological samples may be prepared, either crude or partially (or entirely) purified, in accordance with standard techniques, and used in the methods of the disclosure. Alternatively, biological samples comprising whole cells, e.g. circulating tumor cells (CTCs), can be utilized in assay formats such as immunohistochemistry (IHC), flow cytometry (FC), and immunofluorescence (IF).

### Pharmaceutical Compositions

The disclosure also provides pharmaceutical compositions comprising an antibody, or antigen binding portion thereof, and a pharmaceutically acceptable carrier.

The pharmaceutical compositions comprising antibodies are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies. In another embodiment, the pharmaceutical composition comprises one or more antibodies and one or more prophylactic or therapeutic agents other than antibodies for treating a disorder in which CDCP1 activity is detrimental. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions can be incorporated into pharmaceutical compositions suitable for administration to a subject. Typically, the pharmaceutical composition comprises an antibody or antibody portion and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition.

Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies or the combination of one or more antibodies and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934, 272, 5,874,064, 5,855,913, 5,290, 540, and 4,880,078; and PCT Publication Nos. WO 92/19244, WO 97/32572, WO 97/44013, WO 98/31346, and WO 99/66903, each of which is incorporated herein by reference their entireties. In one embodiment, an antibody, combination therapy, or a composition is administered using Alkermes AIR® pulmonary drug delivery technology (Alkermes, Inc., Cambridge, Mass.). In a specific embodiment, prophylactic or therapeutic agents are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies antagonists is administered locally to the affected area to a subject to prevent, treat, manage, and/or ameliorate a disorder or a symptom thereof. In another embodiment, an effective amount of one or more antibodies is administered locally to the affected area in combination with an effective amount of one or more therapies (e.g., one or more prophylactic or therapeutic agents) other than an antibody of a subject to prevent, treat, manage, and/or ameliorate a disorder or one or more symptoms thereof.

A pharmaceutical composition is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

In some embodiments, the methods of the disclosure comprises oral administration where compositions can be formulated orally in the form of tablets, capsules, cachets, gel caps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the disclosure also provides that one or more of the prophylactic or therapeutic agents, or pharmaceutical compositions is packaged in a hermetically sealed container such as an ampoule or sachette indicating the quantity of the agent. In one embodiment, one or more of the prophylactic or therapeutic agents, or pharmaceutical compositions is supplied as a dry sterilized lyophilized powder or water free concentrate in a hermetically sealed container and can be reconstituted (e.g., with water or saline) to the appropriate concentration for administration to a subject. The disclosed antibodies and/or antigen binding fragments thereof can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The pharmaceutical compositions may include a “therapeutically effective amount” or a “prophylactically effective amount” of an antibody or antigen binding fragments thereof. A “therapeutically effective amount” refers to an amount effective, at dosages and for periods of time necessary, to achieve the desired therapeutic result. A therapeutically effective amount of the antibody or antigen binding fragments thereof may be determined by a person skilled in the art and may vary according to factors such as the disease state, age, sex, and weight of the individual, and the ability of the antibody or antigen binding fragments thereof to elicit a desired response in the individual. A therapeutically effective amount is also one in which any toxic or detrimental effects of the antibody, or antigen binding fragments thereof, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antigen binding fragments thereof is 0.1-20 mg/kg, more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The combination of therapeutic agents discussed herein can be administered concurrently as components of a fusion protein or as a single composition in a pharmaceutically acceptable carrier. Alternatively, a combination of therapeutics can be administered concurrently as separate compositions with each agent in a pharmaceutically acceptable carrier. In another embodiment, the combination of therapeutic agents can be administered sequentially.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. In some aspects, the pharmaceutical composition is administered to a subject to treat cancer. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, may be coated in a material to protect the compound from the action of acids and other natural conditions that may inactivate the compound. A composition of the present disclosure can be administered by a variety of methods known in the art. As will be appreciated by the skilled artisan, the route and/or mode of administration will vary depending upon the desired results. The active compounds can be prepared with carriers that will protect the compound against rapid releases, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

Dosage levels of the active ingredients in the pharmaceutical compositions may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical compositions described herein may be administered in effective amounts. An “effective amount” refers to the amount which achieves a desired reaction or the desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease.

### NUMBERED EMBODIMENTS

The disclosure will be further described in the following embodiments, which do not limit the scope of the disclosure described in the claims.

Embodiment 1. An anti-CDCP1 antibody comprising a heavy chain variable region (VH) and a light chain variable region (VL), wherein:
a) the VH comprises CDR1: SEQ ID NO: 3, CDR2: SEQ ID NO: 4, CDR3: SEQ ID NO: 13, and the VL comprises: CDR1: SEQ ID NO: 8, CDR2: SEQ ID NO: 9, CDR3: SEQ ID NO: 10;
b) the VH comprises CDR1: SEQ ID NO: 3, CDR2: SEQ ID NO: 4, CDR3: SEQ ID NO: 16, and the VL comprises: CDR1: SEQ ID NO: 8, CDR2: SEQ ID NO: 9, CDR3: SEQ ID NO: 10;
c) the VH comprises CDR1: SEQ ID NO: 3, CDR2: SEQ ID NO: 4, CDR3: SEQ ID NO: 19, and the VL comprises: CDR1: SEQ ID NO: 8, CDR2: SEQ ID NO: 9, CDR3: SEQ ID NO: 10;
d) the VH comprises CDR1: SEQ ID NO: 3, CDR2: SEQ ID NO: 4, CDR3: SEQ ID NO: 22, and the VL comprises: CDR1: SEQ ID NO: 8, CDR2: SEQ ID NO: 9, CDR3: SEQ ID NO: 10;
e) the VH comprises CDR1: SEQ ID NO: 3, CDR2: SEQ ID NO: 4, CDR3: SEQ ID NO: 25, and the VL comprises: CDR1: SEQ ID NO: 8, CDR2: SEQ ID NO: 9, CDR3: SEQ ID NO: 10;
f) the VH comprises CDR1: SEQ ID NO: 3, CDR2: SEQ ID NO: 4, CDR3: SEQ ID NO: 28, and the VL comprises: CDR1: SEQ ID NO: 8, CDR2: SEQ ID NO: 9, CDR3: SEQ ID NO: 10; or
g) the VH comprises CDR1: SEQ ID NO: 3, CDR2: SEQ ID NO: 4, CDR3: SEQ ID NO: 31, and the VL comprises: CDR1: SEQ ID NO: 8, CDR2: SEQ ID NO: 9, CDR3: SEQ ID NO: 10.

Embodiment 2. The anti-CDCP1 antibody of Embodiment 1, wherein the antibody comprises:
a) a heavy chain variable (VH) region having an amino acid sequence set forth in SEQ ID NO: 12 and a light chain variable (VL) region having an amino acid sequence as set forth in SEQ ID NO: 7;
b) a VH region having an amino acid sequence as set forth in SEQ ID NO: 15 and a VL region having an amino acid sequence as set forth in SEQ ID NO: 7;
c) a VH region having an amino acid sequence as set forth in SEQ ID NO: 18 and a VL region having an amino acid sequence as set forth in SEQ ID NO: 7;
d) a VH region having an amino acid sequence as set forth in SEQ ID NO: 21 and a VL region having an amino acid sequence as set forth in SEQ ID NO: 7;
e) a VH region having an amino acid sequence as set forth in SEQ ID NO: 24 and a VL region having an amino acid sequence as set forth in SEQ ID NO: 7;
f) a VH region having an amino acid sequence as set forth in SEQ ID NO: 27 and a VL region having an amino acid sequence as set forth in SEQ ID NO: 7; or
g) a VH region having an amino acid sequence as set forth in SEQ ID NO: 30 and a VL region having an amino acid sequence as set forth in SEQ ID NO: 7.

Embodiment 3. The anti-CDCP1 antibody of Embodiment 1, wherein the antibody comprises:
a) a heavy chain having an amino acid sequence as set forth in SEQ ID NO: 11 and a light chain having an amino acid sequence as set forth in SEQ ID NO: 6;
b) a heavy chain having an amino acid sequence as set forth in SEQ ID NO: 14 and a light chain having an amino acid sequence as set forth in SEQ ID NO: 6;
c) a heavy chain having an amino acid sequence as set forth in SEQ ID NO: 17 and a light chain having an amino acid sequence as set forth in SEQ ID NO: 6;
d) a heavy chain having an amino acid sequence as set forth in SEQ ID NO: 20 and a light chain having an amino acid sequence as set forth in SEQ ID NO: 6;
e) a heavy chain having an amino acid sequence as set forth in SEQ ID NO: 23 and a light chain having an amino acid sequence as set forth in SEQ ID NO: 6;
f) a heavy chain having an amino acid sequence as set forth in SEQ ID NO: 26 and a light chain having an amino acid sequence as set forth in SEQ ID NO: 6;
or
g) a heavy chain having an amino acid sequence as set forth in SEQ ID NO: 29 and a light chain having an amino acid sequence as set forth in SEQ ID NO: 6.

Embodiment 4. The anti-CDCP1 antibody according to any one of Embodiments 1 or 2, wherein the antibody comprises a heavy chain constant region as set forth in SEQ ID NO: 32.

Embodiment 5. The anti-CDCP1 antibody according to any one of Embodiments 1 or 2, wherein the antibody comprises a light chain constant region as set forth in SEQ ID NO: 33.

Embodiment 6. The anti-CDCP1 antibody according to any one of Embodiments 1-5, wherein the antibody is a fully human antibody.

Embodiment 7. The anti-CDCP1 antibody according to any one of Embodiments 1-5, wherein the antibody is a chimeric antibody.

Embodiment 8. The anti-CDCP1 antibody according to any one of Embodiments 1-5, wherein the antibody is a humanized antibody.

Embodiment 9. The anti-CDCP1 antibody according to any one of Embodiments 1-5, wherein the antibody is an antibody fragment.

Embodiment 10. The anti-CDCP1 antibody of Embodiment 9, wherein the antibody fragment is selected from the group consisting of: Fab, Fab, F(ab)₂, Fd, Fv, scFv and scFv-Fc fragment, a single-chain antibody, a minibody, and a diabody.

Embodiment 11. An anti-CDCP1 antibody or antigen binding fragment thereof comprising:
a1) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 13 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a2) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 16 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a3) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 19 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a4) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 22 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a5) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 25 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a6) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 28 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a7) no more than 1, 2, or 3 amino acids from SEQ ID NO: 8 (LCDR1), SEQ ID NO: 9 (LCDR2), and SEQ ID NO: 10 (LCDR3);
a8) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 31 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
or
b1) a heavy chain variable (VH) region comprising an amino acid sequence at least 90%, 95%, or 99% identical to SEQ ID NOs: 12, 15, 18, 21, 24, 27, or 30 and
b2) a light chain variable (VL) region comprising an amino acid sequence at least 90%, 95%, or 99% identical to SEQ ID NO: 7.

Embodiment 12. A pharmaceutical composition comprising as an active ingredient, at least one antibody according to any one of Embodiments 1 to 11 and a pharmaceutically acceptable carrier.

Embodiment 13. The pharmaceutical composition according to Embodiment 12 for use in modulating the immune system by inhibiting binding of anti-CDCP1 to CDCP1.

Embodiment 14. The pharmaceutical composition according to any one of Embodiments 12 or 13 for use in treating cancer.

Embodiment 15. A method of treating cancer comprising administering to a subject in need thereof, a pharmaceutical composition according to according to any one of Embodiments 12 to 14.

Embodiment 16. An isolated polynucleotide comprising a sequence encoding an anti-CDCP1 antibody according to according to any one of Embodiments 1 to 11.

Embodiment 17. An isolated polynucleotide encoding a sequence as set forth in any one of SEQ ID NOS: 7, 12, 15, 18, 21, 24, and 27.

Embodiment 18. A vector comprising a polynucleotide according to Embodiment 17.

Embodiment 19. A host cell comprising a polynucleotide according to Embodiment 17, and/or a vector according to Embodiment 18.

Embodiment 20. A method for the production of an anti-CDCP1 antibody according to Embodiment 1, the method comprising culturing the host cell of Embodiment 19.

### EXAMPLES

The following examples are provided to illustrate, but not to limit, the present disclosure.

### Example 1: Generation of Antibodies Against the CDCP1 Extracellular Domain

Experiments were performed to generate fully human antibodies against CDCP1 extracellular domain. The following methods were used in the examples.

### Recombinant CDCP1 Cloning

Human CDCP1 cDNA was purchased from Origene (RC220633, Rockville, MD) and named BBP374. The encoded protein was aligned with GenBank CDCP1 HUMAN sequence and found to differ at two positions: Q525R and D709G.

Ectodomains of human, cynomolgus monkey, rat, and mouse were cloned by either PCR (human) or synthetic genes (monkey, rat, mouse). The synthetic genes were based on GenBank sequences (see Table 3). All DNA sequences were cloned into appropriate CMV-based expression vectors with non-native signal peptides and C-terminal histidine tags for purification.

TABLE 3
Source of CDCP1 Proteins

| | |
|---|---|
| Human | CDCP1_HUMAN |
| Cynomolgus Monkey | XP_005546930 |
| Rat | NP_001100339 |
| Mouse | CDCP1_MOUSE |

The Origene® BBP374 construct was used to express full-length human CDCP1 protein (MI-E836) on the surface of HEK-293 cells. In addition, a series of human CDCP1 constructs were generated to screen antibody binding properties and epitopes (see Table 4).

TABLE 4
CDCP1 cell-surface expression vectors

| Plasmid name | Sequence feature | Comment |
|---|---|---|
| BBP37 | Ml-E836 Q525R D709G (plus | Native CDCP1 |
| | Myc-DDK) | |
| pMSCV/FLCDCP1 | Ml-E836 Q525R D709G (plus | Native CDCP1 |
| | Myc-DDK) | |
| pMSCV/ClvCDCP | K343-E836 Q525R D709G | |
| 1 (N342) | (plus Myc-DKK) | |

A series of CDCP1 constructs were generated to secrete portions of the ectodomain from CHO cells. These plasmids were cloned (see Table 5) to assist with biophysical evaluations.

TABLE 5
CDCP1 ectodomain expression vectors

| Plasmid | | | |
|---|---|---|---|
| Name | Species | Sequence Feature | Comment |
| BBP464 | Human | F30-T667 | Full ectodomain |
| BBP463 | Human | F30-T667 Q525R | Full ectodomain with |
| | | | Q525R SNP |
| BBP477 | Human | F30-T667 R368GS | Impaired R368-K369 |
| | | K369GS Q525R | cleavage |
| BBP464 | Human | F30-R368 | Distal domain |
| BBP465 | Human | K343-T667 Q525R | Proximal domain |
| BBP467 | Human | C221-E544 Q525R | CUB1/CUB2 domain |
| BBP468 | Cynomolgus | F30-T667 | Full monkey |
| | | | ectodomain |
| BBP469 | Rat | S30-T667 | Full rat ectodomain |
| BBP470 | Mouse | S30-T663 | Full mouse ectodomain |

### Transient Expression System

The CDCP1 recombinant proteins and anti-CDCP1 antibodies were expressed in Chinese hamster ovary (CHO) cells using recommended transfection and media components of the ExpiCHO system (Invitrogen, Carlsbad, CA). Cell culture supernatants were harvested 14 days post-transfection, centrifuged, and filtered (0.22 μm) prior to purification.

### Purification of Recombinant His-Tagged Proteins

Conditioned medium from CHO cell cultures was clarified, filtered, and loaded onto an AKTAprime plus system with a 5 mL HisTrap™ FF column (GE Healthcare). Fractions were collected, analyzed by SDS-PAGE, pooled, and dialyzed against PBS.

### Antibody Purification

Conditioned medium from CHO cell cultures was clarified, filtered, and purified by loading onto an AKTA pure system with a 5 mL MabSelect SuRe® column (GE Healthcare). Antibodies were eluted with 100 mM glycine, pH 3.5 and neutralized with IM Tris-Cl, pH 8.5.

### Recombinant Antibody Analyses

### Concentration:

Concentration of recombinant antibodies was determined on a Fortebio Octet using Protein A tips and a human IgG1 antibody for the standard curve.

### Purity Testing by SDS-PAGE:

Purity testing was performed by sodium dodecyl sulfate polyacrylamide gel electrophoresis (SDS-PAGE) of reduced and non-reduced samples. Samples (10 μg) were mixed with loading buffer (+/−beta-mercaptoethanol), heated, and electrophoresed on a 4-20% gel (Invitrogen). Bands were visualized by Coomassie InstantBlue® (Expedeon) staining.

### Purity Testing by Endotoxin:

Endotoxin concentrations were measured by the Limulus amoebocyte lysate (LAL) kinetic turbidometric method using the Endosafe-PTS® system (Charles River Laboratories).

Purity testing by HPLC-SEC: Samples were screened for aggregation or other forms of antibody on a 1260 Infinity System (Agilent) with a TSKgel UltraSW® Aggregate Guard column and HPLC column (Tosoh Bioscience). Samples and standards were detected by absorbance at 280 nm. Comparison against the standard curve provided the molar mass of sample components.

### Affinity:

The affinity of antibodies to various recombinant CDCP1 molecules was determined on an Octet Red (Pall, ForteBio) instrument. After loading reagents into a 96-well plate, the Octet Red with Protein A-conjugated biosensors was programmed as follows: 30 seconds for baseline #1; 120 seconds to immobilize the antibody; 30 seconds for baseline #2; 300 seconds for association of antibody to recombinant CDCP1; and 300-600 seconds for dissociation of recombinant CDCP1 from the antibody.

### Epitope Binning:

Binding competition among different antibodies was determined using a real-time, interferometry assay on an Octet Red (Pall, ForteBio) instrument with Protein A-conjugated biosensors. To assess whether two antibodies competed for binding to a recombinant CDCP1 protein, the assay was performed as follows. Protein A biosensors were first submerged into wells containing 20 μg/mL of individual monoclonal antibodies for 10 minutes. Following the capture step, the biosensors were dipped briefly (30 sec) into buffer and then any unoccupied sites on the biosensor were saturated by submerging them for 5 minutes into wells containing 200 μg/mL of an irrelevant monoclonal antibody. The Octet biosensors were then dipped briefly (30 sec) in buffer before immersion for 5 minutes into wells containing recombinant CDCP1. The biosensors were dipped briefly (30 sec) in buffer before immersion for 5 minutes into wells containing a second recombinant antibody.

For the control case where the second antibody was the same as the first, there was no increase in signal, because there was no additional binding to the recombinant target. For the control case where buffer was used instead of the first antibody, no recombinant target bound the non-quenching antibody on the biosensor and no second antibody bound the biosensor. For cases where a boost in signal was seen with the second antibody, the two antibodies were determined not to compete. For cases where no boost in signal was seen with the second antibody, the two antibodies were determined to compete for binding.

### Immunofluorescence (IF) Based High Content Screening (HCS)

High content immunofluorescence was used to identify wells that contain immunoglobulin that preferentially bound CDCP1. Briefly, HCT116 cells (CDCP1+) and MCF7 cells (CDCP1-) seeded 24 hours before the assay were incubated for 45 minutes at 37° C. with hybridoma supernatant diluted 2-fold in DMEM+10% fetal bovine serum (FBS). After incubation, cells were fixed in 4% formaldehyde, washed with PBS, permeabilized with 0.3% Triton®-X-100, and labeled with anti-human Alexa® 488 secondary antibodies for 1 hour at room temperature. Unbound secondary antibody was removed with PBS washes, and cells were stained with DNA dye (propidium iodide and Hoechst 33342).

Potential hits were initially identified via low-resolution, high throughput screening using a TTP Labtech Acumen eX3® (TTP Labtech, Cambridge, MA), quantifying the fluorescence differential for each sample on both positive and negative cell lines. Those hits were subsequently verified and the subcellular localization of each sample was characterized using a Thermo ArrayScan VTi® (Thermo Fisher Scientific, Waltham, MA) to obtain high-resolution images of both cell lines. Wells containing immunoglobulin that preferentially bound the CDCP1 were analyzed again on 293T cells or 293T cells over-expressing full length (FL) CDCP1 and cleaved-CDCP1 to confirm CDCP1 specificity and to determine epitope localization.

### Determination of Epitope Localization of CDCP1 Antibodies

Two proteolytic cleavage sites (R368 and N342) in the CDCP1 extracellular domain were identified using mass spectrometry technology. 293T cells stably transfected with DNA constructs expressing full length (FL) CDCP1 and cleaved (Clv) CDCP1 (AA343-836) expresses these proteins on the cell surface.

### Results

Recombinant human IgG1 antibodies against CDCP1 were prepared. Biological characterization of these antibodies include target internalization/degradation capabilities and matripase-mediated R368 cleavage blockade. Biophysical characterization of these antibodies include: cross-reactivity against human and Cynomolgus CDCP1, epitope competition binning, aggregation, degradation, stickiness and sequence liability. Seven recombinant monoclonal human antibodies were selected for epitope binning assay, kinetic analysis against human and Cynomolgus CDCP1. The sequences of these antibodies are provided in FIGS. 1A-1H and Table 6.

TABLE 6
Listing of various anti-CDCP1 antibodies and variant thereof of the present
disclosure.

| | | SEQ |
|---|---|---|
| | | ID |
| | SEQUENCE | NQ: |
| Heavy Chain | EVQLLESGGGLVQPGGSLRLSCAASGFTFSNYAMNWVRQAPGKGLEWVSAIS | 1 |
| (Reference) | 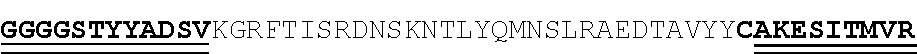 | |
| | 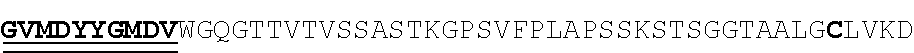 | |
| | YFPEPVTVSWNSGALTSGVHTFPAVLQSSGLYSLSSVVTVPSSSLGTQTYICNVN | |
| | HKPSNTKVDKRVEPKSCDKTHTCPPCPAPELLGGPSVFLFPPKPKDTLMISRTPEV | |
| | TCVVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSREEMTKNQVSLT | |
| | CLVKGFYPSDIAVEWESNGQPENNYKTTPPVLDSDGSFFLYSKLTVDKSRWQQG | |
| | NVFSCSVMHEALHNHYTQKSL | |
| | SLSPG | |
| Heavy | ASTKGPSVFPLAPSSKSTSGGTAALGCLVKDYFPEPVTVSWNSGALTSGVHTFPA | 32 |
| Constant | VLQSSGLYSLSSVVTVPSSSLGTQTYICNVNHKPSNTKVDKRVEPKSCDKTHTCPP | |
| | CPAPELLGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENN | |
| | YKTTPPVLDSDGSFFLYSKLTVDKSRWQQGNVFSCSVMHEALHNHYTQKSL | |
| | SLSPG | |
| VH | EVQLLESGGGLVQPGGSLRLSCAASGFTFSNYAMNWVRQAPGKGLEWVSAIS | 2 |
| | 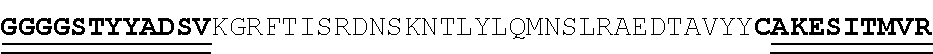 | |
| | 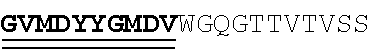 | |
| VH CDR1 | GFTFSNYA | 3 |
| VH CDR2 | AISGGGGSTYYADSV | 4 |
| VH CDR3 | 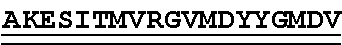 | 5 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| Light | RTVAAPSVFIFPPSDEQLKSGTASVVCLLNNFYPREAKVQWKVDNALQSGNSQE | 33 |
| Constant | SVTEQDSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQS | 9 |
| VL CDR3 | QKYNSAP | 10 |
| Heavy Chain | EVQLLESGGGLVQPGGSLRLSCAASGFTFSNYAMNWVRQAPGKGLEWVSAIS | 11 |
| (M103F) | 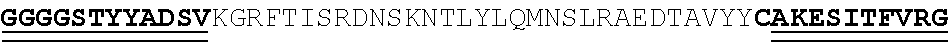 | |
| | 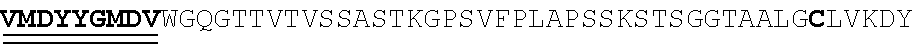 | |
| | FPEPVTVSWNSGALTSGVHTFPAVLQSSGLYSLSSVVTVPSSSLGTQTYICNVNH | |
| | KPSNTKVDKRVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKSRWQQG | |
| | NVFSCSVMHEALHNHYTQKSLSLSPG | |
| VH | EVQLLESGGGLVQPGGSLRLSCAASGFTFSNYAMNWVRQAPGKGLEWVSAIS | 12 |
| | 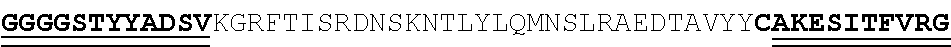 | |
| | 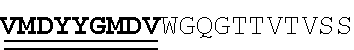 | |
| VH CDR1 | GFTFSNYA | 3 |
| VH CDR2 | AISGGGGSTYYADSV | 4 |
| VH CDR3 | 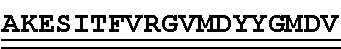 | 13 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |
| Heavy Chain | EVQLLESGGGLVQPGGSLRLSCAASGFTFSNYAMNWVRQAPGKGLEWVSAIS | 14 |
| (M103F | 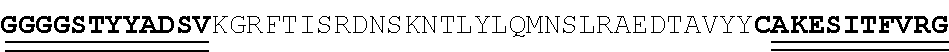 | |
| M108L) | 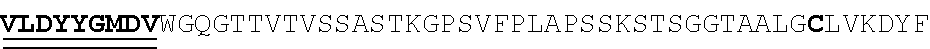 | |
| | PEPVTVSWNSGALTSGVHTFPAVLQSSGLYSLSSVVTVPSSSLGTQTYICNVNHK | |
| | PSNTKVDKRVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSLTCLV | |
| | KGFYPSDIAVEWESNGQPENNYKTTPPVLDSDGSFFLYSKLTVDKSRWQQGNV | |
| | FSCSVMHEALHNHYTQKSLSLSPG | |
| VH | EVQLLESGGGLVQPGGSLRLSCAASGFTFSNYAMNWVRQAPGKGLEWVSAIS | 15 |
| | 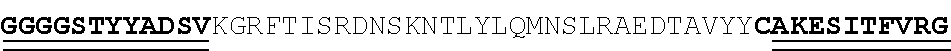 | |
| | 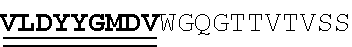 | |
| VH CDR1 | GFTFSNYA | 3 |
| VH CDR2 | AISGGGGSTYYADSV | 4 |
| VH CDR3 | 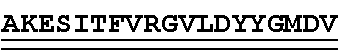 | 16 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |
| Heavy Chain | EVQLLESGGGLVQPGGSLRLSCAASGFTFSNYAMNWVRQAPGKGLEWVSAIS | 17 |
| (M103K) | 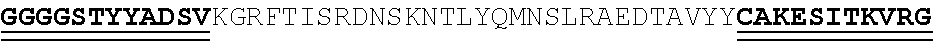 | |
| | 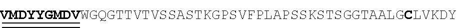 | |
| | FPEPVTVSWNSGALTSGVHTFPAVLQSSGLYSLSSVVTVPSSSLGTQTYICNVNH | |
| | KPSNTKVDKRVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKSRWQQG | |
| | NVFSCSVMHEALHNHYTQKSLSLSPG | |
| VH | EVQLLESGGGLVQPGGSLRLSCAASGFTFSNYAMNWVRQAPGKGLEWVSAIS | 18 |
| | 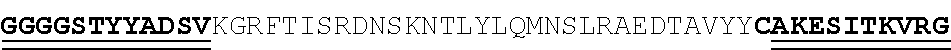 | |
| | 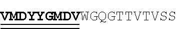 | |
| VH CDR1 | GFTFSNYA | 3 |
| VH CDR2 | AISGGGGSTYYADSV | 4 |
| VH CDR3 | 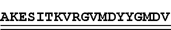 | 19 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |
| Heavy Chain | EVQLLESGGGLVQPGGSLRLSCAASGFTFSNYAMNWVRQAPGKGLEWVSAIS | 20 |
| (M103K | 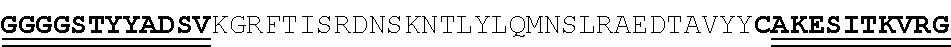 | |
| M108L) | 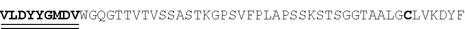 | |
| | PEPVTVSWNSGALTSGVHTFPAVLQSSGLYSLSSVVTVPSSSLGTQTYICNVNHK | |
| | PSNTKVDKRVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSLTCLV | |
| | KGFYPSDIAVEWESNGQPENNYKTTPPVLDSDGSFFLYSKLTVDKSRWQQGNV | |
| | FSCSVMHEALHNHYTQKSLSLSPG | |
| VH | EVQLLESGGGLVQPGGSLRLSCAASGFTFSNYAMNWVRQAPGKGLEWVSAIS | 21 |
| | 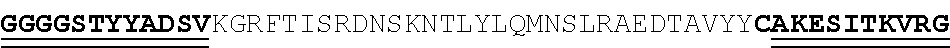 | |
| | 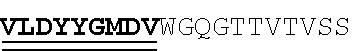 | |
| VH CDR1 | GFTESNYA | 3 |
| VH CDR2 | AISGGGGSTYYADSV | 4 |
| VH CDR3 | 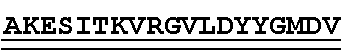 | 22 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |
| Heavy Chain | EVQLLESGGGLVQPGGSLRLSCAASGFTFSNYAMNWVRQAPGKGLEWVSAIS | 23 |
| (M103L) | 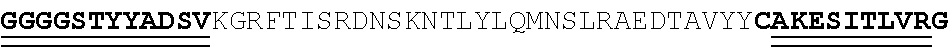 | |
| | 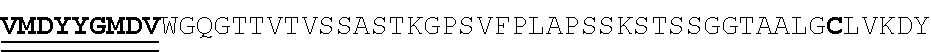 | |
| | FPEPVTVSWNSGALTSGVHTFPAVLQSSGLYSLSSVVTVPSSSLGTQTYICNVNH | |
| | KPSNTKVDKRVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKSRWQQG | |
| | NVFSCSVMHEALHNHYTQKSLSLSPG | |
| VH | EVQLLESGGGLVQPGGSLRLSCAASGFTFSNYAMNWVRQAPGKGLEWVSAIS | 24 |
| | 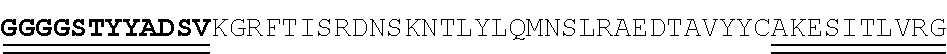 | |
| | 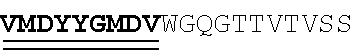 | |
| VH CDR1 | GFTFSNYA | 3 |
| VH CDR2 | AISGGGGSTYYADSV | 4 |
| VH CDR3 | 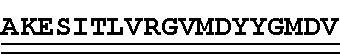 | 25 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |
| Heavy Chain | EVQLLESGGGLVQPGGSLRLSCAASGFTFSNYAMNWVRQAPGKGLEWVSAIS | 26 |
| (M103L | 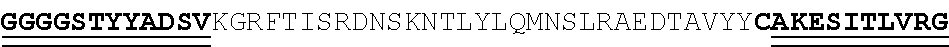 | |
| M108L) | 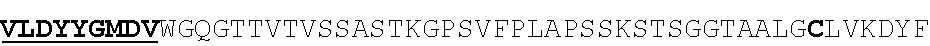 | |
| | PEPVTVSWNSGALTSGVHTFPAVLQSSGLYSLSSVVTVPSSSLGTQTYICNVNHK | |
| | PSNTKVDKRVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSLTCLV | |
| | KGFYPSDIAVEWESNGQPENNYKTTPPVLDSDGSFFLYSKLTVDKSRWQQGNV | |
| | FSCSVMHEALHNHYTQKSLSLSPG | |
| VH | EVQLLESGGGLVQPGGSLRLSCAASGFTFSNYAMNWVRQAPGKGLEWVSAIS | 27 |
| | 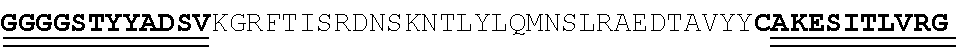 | |
| | 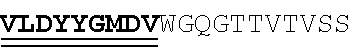 | |
| VH CDR1 | GFTFSNYA | 3 |
| VH CDR2 | AISGGGGSTYYADSV | 4 |
| VH CDR3 | 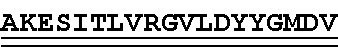 | 28 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |
| Heavy Chain | VQLLESGGGLVQPGGSLRLSCAASGFTFSNYAMNWVRQAPGKGLEWVSAISG | 29 |
| (M108L) | 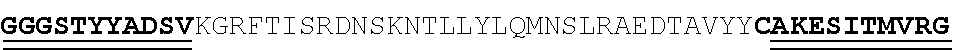 | |
| | 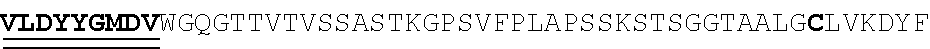 | |
| | PEPVTVSWNSGALTSGVHTFPAVLQSSGLYSLSSVVTVPSSSLGTQTYICNVNHK | |
| | PSNTKVDKRVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSLTCLV | |
| | KGFYPSDIAVEWESNGQPENNYKTTPPVLDSDGSFFLYSKLTVDKSRWQQGNV | |
| | FSCSVMHEALHNHYTQKSLSLSPG | |
| VH | EVQLLESGGGLVQPGGSLRLSCAASGFTFSNYAMNWVRQAPGKGLEWVSAIS | 30 |
| | 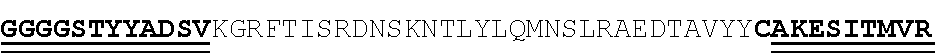 | |
| | 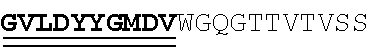 | |
| VH CDR1 | GFTFSNYA | 3 |
| VH CDR2 | AISGGGGSTYYADSV | 4 |
| VH CDR3 | 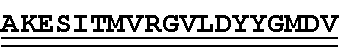 | 31 |
| Light Chain | DIQLTQSPSFLSASVGDRVTITCRASQGISSYLAWYQQKPGKAPKLLIYAASTLQG | 6 |
| | GVPSRFSGSGSGTEFTLTISSLQPEDFATYYCQHLNRFPRTFGQGTKVEIKRTVAA | |
| | PSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC | |
| VL | DIQLTQSPSFLSASVGDRVTITCRASQGISSYLAWYQQKPGKAPKLLIYAASTLQG | 7 |
| | GVPSRFSGSGSGTEFTLTISSLQPEDFATYYCQHLNRFPRTFGQGTKVEIK | |
| VL CDR1 | RASQGISSYLA | 8 |
| VL CDR2 | AASTLQG | 9 |
| VL CDR3 | QHLNRFPRT | 10 |

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims. Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

What is claimed is:

1. An anti-CDCP1 antibody comprising a heavy chain variable region (VH) and a light chain variable region (VL), wherein:
a) the VH comprises CDR1: SEQ ID NO: 3, CDR2: SEQ ID NO: 4, CDR3: SEQ ID NO: 13, and the VL comprises: CDR1: SEQ ID NO: 8, CDR2: SEQ ID NO: 9, CDR3: SEQ ID NO: 10;
b) the VH comprises CDR1: SEQ ID NO: 3, CDR2: SEQ ID NO: 4, CDR3: SEQ ID NO: 16, and the VL comprises: CDR1: SEQ ID NO: 8, CDR2: SEQ ID NO: 9, CDR3: SEQ ID NO: 10;
c) the VH comprises CDR1: SEQ ID NO: 3, CDR2: SEQ ID NO: 4, CDR3: SEQ ID NO: 19, and the VL comprises: CDR1: SEQ ID NO: 8, CDR2: SEQ ID NO: 9, CDR3: SEQ ID NO: 10;
d) the VH comprises CDR1: SEQ ID NO: 3, CDR2: SEQ ID NO: 4, CDR3: SEQ ID NO: 22, and the VL comprises: CDR1: SEQ ID NO: 8, CDR2: SEQ ID NO: 9, CDR3: SEQ ID NO: 10;
e) the VH comprises CDR1: SEQ ID NO: 3, CDR2: SEQ ID NO: 4, CDR3: SEQ ID NO: 25, and the VL comprises: CDR1: SEQ ID NO: 8, CDR2: SEQ ID NO: 9, CDR3: SEQ ID NO: 10;
f) the VH comprises CDR1: SEQ ID NO: 3, CDR2: SEQ ID NO: 4, CDR3: SEQ ID NO: 28, and the VL comprises: CDR1: SEQ ID NO: 8, CDR2: SEQ ID NO: 9, CDR3: SEQ ID NO: 10; or
g) the VH comprises CDR1: SEQ ID NO: 3, CDR2: SEQ ID NO: 4, CDR3: SEQ ID NO: 31, and the VL comprises: CDR1: SEQ ID NO: 8, CDR2: SEQ ID NO: 9, CDR3: SEQ ID NO: 10.

2. The anti-CDCP1 antibody of claim 1, wherein the antibody comprises:
a) a heavy chain variable (VH) region having an amino acid sequence set forth in SEQ ID NO: 12 and a light chain variable (VL) region having an amino acid sequence as set forth in SEQ ID NO: 7;
b) a VH region having an amino acid sequence as set forth in SEQ ID NO: 15 and a VL region having an amino acid sequence as set forth in SEQ ID NO: 7;
c) a VH region having an amino acid sequence as set forth in SEQ ID NO: 18 and a VL region having an amino acid sequence as set forth in SEQ ID NO: 7;
d) a VH region having an amino acid sequence as set forth in SEQ ID NO: 21 and a VL region having an amino acid sequence as set forth in SEQ ID NO: 7;
e) a VH region having an amino acid sequence as set forth in SEQ ID NO: 24 and a VL region having an amino acid sequence as set forth in SEQ ID NO: 7;
f) a VH region having an amino acid sequence as set forth in SEQ ID NO: 27 and a VL region having an amino acid sequence as set forth in SEQ ID NO: 7; or
g) a VH region having an amino acid sequence as set forth in SEQ ID NO: 30 and a VL region having an amino acid sequence as set forth in SEQ ID NO: 7.

3. The anti-CDCP1 antibody of claim 1, wherein the antibody comprises:
a) a heavy chain having an amino acid sequence as set forth in SEQ ID NO: 11 and a light chain having an amino acid sequence as set forth in SEQ ID NO: 6;
b) a heavy chain having an amino acid sequence as set forth in SEQ ID NO: 14 and a light chain having an amino acid sequence as set forth in SEQ ID NO: 6;
c) a heavy chain having an amino acid sequence as set forth in SEQ ID NO: 17 and a light chain having an amino acid sequence as set forth in SEQ ID NO: 6;
d) a heavy chain having an amino acid sequence as set forth in SEQ ID NO: 20 and a light chain having an amino acid sequence as set forth in SEQ ID NO: 6;
e) a heavy chain having an amino acid sequence as set forth in SEQ ID NO: 23 and a light chain having an amino acid sequence as set forth in SEQ ID NO: 6;
f) a heavy chain having an amino acid sequence as set forth in SEQ ID NO: 26 and a light chain having an amino acid sequence as set forth in SEQ ID NO: 6; or
g) a heavy chain having an amino acid sequence as set forth in SEQ ID NO: 29 and a light chain having an amino acid sequence as set forth in SEQ ID NO: 6.

4. The anti-CDCP1 antibody according to any one of claim 1 or 2, wherein the antibody comprises a heavy chain constant region as set forth in SEQ ID NO: 32.

5. The anti-CDCP1 antibody according to any one of claim 1 or 2, wherein the antibody comprises a light chain constant region as set forth in SEQ ID NO: 33.

6. The anti-CDCP1 antibody according to any one of claims 1-5, wherein the antibody is a fully human antibody.

7. The anti-CDCP1 antibody according to any one of claims 1-5, wherein the antibody is a chimeric antibody.

8. The anti-CDCP1 antibody according to any one of claims 1-5, wherein the antibody is a humanized antibody.

9. The anti-CDCP1 antibody according to any one of claims 1-5, wherein the antibody is an antibody fragment.

10. The anti-CDCP1 antibody of claim 9, wherein the antibody fragment is selected from the group consisting of: Fab, Fab, F(ab)2, Fd, Fv, scFv and scFv-Fc fragment, a single-chain antibody, a minibody, and a diabody.

11. An anti-CDCP1 antibody or antigen binding fragment thereof comprising:
a1) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 13 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a2) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 16 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a3) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 19 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a4) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 22 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a5) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 25 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a6) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 28 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
a7) no more than 1, 2, or 3 amino acids from SEQ ID NO: 8 (LCDR1), SEQ ID NO: 9 (LCDR2), and SEQ ID NO: 10 (LCDR3);
a8) heavy chain CDRs comprising amino acid sequences that differ by no more than 1, 2, or 3 amino acids from SEQ ID NO: 3 (HCDR1), SEQ ID NO: 4 (HCDR2), and SEQ ID NO: 31 (HCDR3); and light chain CDRs comprising amino acid sequences that differ by no more than 1, 2, or 3 amino acids from SEQ ID NO: 8 (LCDR1), SEQ ID NO: 9 (LCDR2), and SEQ ID NO: 10 (LCDR3);
or
b1) a heavy chain variable (VH) region comprising an amino acid sequence at least 90%, 95%, or 99% identical to SEQ ID NOs: 12, 15, 18, 21, 24, 27, or 30 and
b2) a light chain variable (VL) region comprising an amino acid sequence at least 90%, 95%, or 99% identical to SEQ ID NO: 7.

12. A pharmaceutical composition comprising as an active ingredient, at least one antibody according to any one of claims 1 to 11 and a pharmaceutically acceptable carrier.

13. The pharmaceutical composition according to claim 12 for use in modulating the immune system by inhibiting binding of anti-CDCP1 to CDCP1.

14. The pharmaceutical composition according to any one of claim 12 or 13 for use in treating cancer.

15. A method of treating cancer comprising administering to a subject in need thereof, a pharmaceutical composition according to any one of claims 12-14.

16. An isolated polynucleotide comprising a sequence encoding an anti-CDCP1 antibody according to anyone of claims 1-11.

17. An isolated polynucleotide encoding a sequence as set forth in any one of SEQ ID NOS: 7, 12, 15, 18, 21, 24, and 27.

18. A vector comprising a polynucleotide according to claim 17.

19. A host cell comprising a polynucleotide according to claim 17, and/or a vector according to claim 18.

20. A method for the production of an anti-CDCP1 antibody according to claim 1, the method comprising culturing the host cell of claim 19.


SEQUENCE LISTING (33 sequences)

SEQ ID NO: 1 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITMVRGVMDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITMVRGVMDYYGMDVWGQGTTVTVSS

SEQ ID NO: 3 - protein, 8 aa, Homo sapiens
GFTFSNYA

SEQ ID NO: 4 - protein, 15 aa, Homo sapiens
AISGGGGSTYYADSV

SEQ ID NO: 5 - protein, 19 aa, Homo sapiens
AKESITMVRGVMDYYGMDV

SEQ ID NO: 6 - protein, 214 aa, Homo sapiens
DIQLTQSPSFLSASVGDRVTITCRASQGISSYLAWYQQKPGKAPKLLIYAASTLQGGVPSRFSGSGSGTEFTLTISSLQPEDFATYYCQHLNRFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Homo sapiens
DIQLTQSPSFLSASVGDRVTITCRASQGISSYLAWYQQKPGKAPKLLIYAASTLQGGVPSRFSGSGSGTEFTLTISSLQPEDFATYYCQHLNRFPRTFGQGTKVEIK

SEQ ID NO: 8 - protein, 11 aa, Homo sapiens
RASQGISSYLA

SEQ ID NO: 9 - protein, 7 aa, Homo sapiens
AASTLQS

SEQ ID NO: 10 - protein, 7 aa, Homo sapiens
QKYNSAP

SEQ ID NO: 11 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITFVRGVMDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITFVRGVMDYYGMDVWGQGTTVTVSS

SEQ ID NO: 13 - protein, 19 aa, Homo sapiens
AKESITFVRGVMDYYGMDV

SEQ ID NO: 14 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITFVRGVLDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITFVRGVLDYYGMDVWGQGTTVTVSS

SEQ ID NO: 16 - protein, 19 aa, Homo sapiens
AKESITFVRGVLDYYGMDV

SEQ ID NO: 17 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITKVRGVMDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITKVRGVMDYYGMDVWGQGTTVTVSS

SEQ ID NO: 19 - protein, 19 aa, Homo sapiens
AKESITKVRGVMDYYGMDV

SEQ ID NO: 20 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITKVRGVLDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITKVRGVLDYYGMDVWGQGTTVTVSS

SEQ ID NO: 22 - protein, 19 aa, Homo sapiens
AKESITKVRGVLDYYGMDV

SEQ ID NO: 23 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITLVRGVMDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITLVRGVMDYYGMDVWGQGTTVTVSS

SEQ ID NO: 25 - protein, 19 aa, Homo sapiens
AKESITLVRGVMDYYGMDV

SEQ ID NO: 26 - protein, 455 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITLVRGVLDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITLVRGVLDYYGMDVWGQGTTVTVSS

SEQ ID NO: 28 - protein, 19 aa, Homo sapiens
AKESITLVRGVLDYYGMDV

SEQ ID NO: 29 - protein, 454 aa, Homo sapiens
VQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITMVRGVLDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 126 aa, Homo sapiens
EVQLLESGGGLVQPGGSLRLSCAASGFTFSNYAMNWVRQAPGKGLEWVSAISGGGGSTYYADSVKGRFTISRDNSKNTLYLQMNSLRAEDTAVYYCAKESITMVRGVLDYYGMDVWGQGTTVTVSS

SEQ ID NO: 31 - protein, 19 aa, Homo sapiens
AKESITMVRGVLDYYGMDV

SEQ ID NO: 32 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 107 aa, Homo sapiens [sequence omitted]